SEQUENCE LISTING (248 sequences)

SEQ ID NO: 1 - DNA, 74 bp, Artificial Sequence
  Synthetic PCR spike-in sequence
  misc_feature (36)..(37): Diribonucleotides
ggagctgtcgttcactctgtctcggagcttacagtautgacactcaatcggtcgcgtagatcggaagagcacac

SEQ ID NO: 2 - DNA, 20 bp, Homo sapiens
  misc_feature (1)..(1): where ′n′ means any nucleotide-a, g, c or t
  misc_feature (5)..(5): where ′n′ means any nucleotide-a, g, c or t
  misc_feature (8)..(8): where ′n′ means any nucleotide-a, g, c or t
  misc_feature (10)..(10): where ′n′ means any nucleotide-a, g, c or t
  misc_feature (16)..(16): where ′n′ means any nucleotide-a, g, c or t
  misc_feature (18)..(18): where ′n′ means any nucleotide-a, g, c or t
ngccnatnanccctgngnct

SEQ ID NO: 3 - DNA, 37 bp, Candida albicans
  misc_feature (1)..(37): Ligation probe nameCA_18804_P13L_3P
  misc_feature (36)..(37): Diribonucleotides
ggagctgtcgttcactcttaagttcagcgggtagtcc

SEQ ID NO: 4 - DNA, 37 bp, Candida albicans
  misc_feature (1)..(37): Ligation probe name CA-18804_P13L_5P
  misc_feature (1)..(1): 5′ phosphorylated
tacctgatttgaggtcaaagagatcggaagagcacac

SEQ ID NO: 5 - DNA, 37 bp, Candida albicans
  misc_feature (1)..(37): Ligation probe name CA-18804__P8L_3P
  misc_feature (36)..(37): Diribonucleotides
ggagctgtcgttcactccaaacaggcatgccctccgg

SEQ ID NO: 6 - DNA, 37 bp, Candida albicans
  misc_feature (1)..(37): Ligation probe name CA-18804__P8L_5P
  misc_feature (1)..(1): 5′ phosphorylated
aataccagagggcgcaatgtagatcggaagagcacac

SEQ ID NO: 7 - DNA, 37 bp, Candida albicans
  misc_feature (1)..(37): Ligation probe name CA-18804__P2L_3P
  misc_feature (36)..(37): Diribonucleotides
ggagctgtcgttcactcagagagcaacatgcaaaaua

SEQ ID NO: 8 - DNA, 37 bp, Candida albicans
  misc_feature (1)..(37): Ligation probe name CA-18804__P2L_5P
  misc_feature (1)..(1): 5′ phosphorylated
ccaagtctgatctcaagcccagatcggaagagcacac

SEQ ID NO: 9 - DNA, 37 bp, Candida albicans
  misc_feature (1)..(37): Ligation probe name CA-18804_18S_P3L_3P
  misc_feature (36)..(37): Diribonucleotides
ggagctgtcgttcactctcggctgggtccagtacgca

SEQ ID NO: 10 - DNA, 37 bp, Candida albicans
  misc_feature (1)..(37): CA-18804_18S_P3L_5P
  misc_feature (1)..(1): 5′ phosphorylated
tcaaaaagatggaccggccaagatcggaagagcacac

SEQ ID NO: 11 - DNA, 37 bp, Candida albicans; identical to SEQ ID NO: 3
  misc_feature (1)..(37): Ligation probe name C_albicans_ATCC_18804_ITS_3P
  misc_feature (36)..(37): Diribonucleotides

SEQ ID NO: 12 - DNA, 37 bp, Candida albicans; identical to SEQ ID NO: 4
  misc_feature (1)..(37): Ligation probe name C_albicans_ATCC_18804_ITS_5P
  misc_feature (36)..(37): 5′ phosphorylated

SEQ ID NO: 13 - DNA, 37 bp, Candida albicans; identical to SEQ ID NO: 5
  misc_feature (1)..(37): Ligation probe name C_albicans_ATCC_18804_ITS_3P
  misc_feature (36)..(37): Diribonucleotides

SEQ ID NO: 14 - DNA, 37 bp, Candida albicans; identical to SEQ ID NO: 6
  misc_feature (1)..(37): C_albicans_ATCC_18804_ITS_5P
  misc_feature (1)..(1): 5′ phosphorylated

SEQ ID NO: 15 - DNA, 37 bp, Cryptococcus neoformans
  misc_feature (1)..(37): CN-32045__P7L_3P
  misc_feature (36)..(37): Diribonucleotides
ggagctgtcgttcactcttcatgactctcaaacaggc

SEQ ID NO: 16 - DNA, 37 bp, Cryptococcus neoformans
  misc_feature (1)..(37): Ligation probe name CN-32045__P7L_5P
  misc_feature (1)..(1): 5′ phosphorylated
atgcccttcggaataccaaaagatcggaagagcacac

SEQ ID NO: 17 - DNA, 37 bp, Cryptococcus neoformans
  misc_feature (1)..(37): Ligation probe name CN-32045__P1L_3P
  misc_feature (36)..(37): Diribonucleotides
ggagctgtcgttcactctaccctattccaagggacuu

SEQ ID NO: 18 - DNA, 37 bp, Cryptococcus neoformans
  misc_feature (1)..(37): Ligation probe name CN-32045__P1L_5P
  misc_feature (1)..(1): 5′ phosphorylated
agacacggtccagcacggaaagatcggaagagcacac

SEQ ID NO: 19 - DNA, 37 bp, Cryptococcus neoformans
  misc_feature (1)..(37): Ligation probe name CN-32045__P2L_3P
  misc_feature (36)..(37): Diribonucleotides
ggagctgtcgttcactcgaactggctgaacccaatag

SEQ ID NO: 20 - DNA, 37 bp, Cryptococcus neoformans
  misc_feature (1)..(37): Ligation probe name CN-32045__P2L_5P
  misc_feature (1)..(1): 5′ phosphorylated
acacgactgacttcaatcgtagatcggaagagcacac

SEQ ID NO: 21 - DNA, 37 bp, Cryptococcus neoformans
  misc_feature (1)..(37): Ligation probe name CN-32045_18S_P3L_3P
  misc_feature (36)..(37): Diribonucleotides
ggagctgtcgttcactcggtccagcaagacagtgcac

SEQ ID NO: 22 - DNA, 37 bp, Cryptococcus neoformans
  misc_feature (1)..(37): Ligation probe name CN-32045_18S_P3L_5P
  misc_feature (1)..(1): 5′ phosphorylated
tccgtgaggaggaccgcctcagatcggaagagcacac

SEQ ID NO: 23 - DNA, 37 bp, Cryptococcus neoformans
  misc_feature (1)..(37): Ligation probe name CN-32045_18S_P4L_3P
  misc_feature (36)..(37): Diribonucleotides
ggagctgtcgttcactccatacgaccagccaaagccg

SEQ ID NO: 24 - DNA, 37 bp, Cryptococcus neoformans
  misc_feature (1)..(37): Ligation probe name CN-32045_18S_P4L_5P
  misc_feature (1)..(1): 5′ phosphorylated
gcctgactatttagcaggttagatcggaagagcacac

SEQ ID NO: 25 - DNA, 37 bp, Cryptococcus neoformans; identical to SEQ ID NO: 15
  misc_feature (1)..(37): Ligation probe name C_neoformans_ATCC_32045_ITS_3P
  misc_feature (36)..(37): Diribonucleotides

SEQ ID NO: 26 - DNA, 37 bp, Cryptococcus neoformans; identical to SEQ ID NO: 16
  misc_feature (1)..(37): Ligation probe name C_neoformans_ATCC_32045_ITS_5P
  misc_feature (1)..(1): 5′ phosphorylated

SEQ ID NO: 27 - DNA, 37 bp, Haemophilus influenzae
  misc_feature (1)..(37): Ligation probe name HI-10211_P33L_3P
  misc_feature (36)..(37): Diribonucleotides
ggagctgtcgttcactcctccctaaagtactctaguu

SEQ ID NO: 28 - DNA, 37 bp, Haemophilus influenzae
  misc_feature (1)..(37): Ligation probe name HI-10211_P33L_5P
  misc_feature (1)..(1): 5′ phosphorylated
acccagtctgaaatgcaattagatcggaagagcacac

SEQ ID NO: 29 - DNA, 37 bp, Haemophilus influenzae
  misc_feature (1)..(37): Ligation probe name HI-10211_P42L_3P
  misc_feature (36)..(37): Diribonucleotides
ggagctgtcgttcactcgagtaggtaaggttcttcgc

SEQ ID NO: 30 - DNA, 37 bp, Haemophilus influenzae
  misc_feature (1)..(37): Ligation probe name HI-10211_P42L_5P
  misc_feature (1)..(1): 5′ phosphorylated
gttgcatcgaattaaaccacagatcggaagagcacac

SEQ ID NO: 31 - DNA, 37 bp, Haemophilus influenzae
  misc_feature (1)..(37): Ligation probe name HI-10211_P5L_3P
  misc_feature (36)..(37): Diribonucleotides
ggagctgtcgttcactcccccctccataagccagauu

SEQ ID NO: 32 - DNA, 37 bp, Haemophilus influenzae
  misc_feature (1)..(37): Ligation probe name HI-10211_P5L_5P
cccaagcattactcacccgtagatcggaagagcacac

SEQ ID NO: 33 - DNA, 37 bp, Human cytomegalovirus
  misc_feature (1)..(37): Ligation probe name CMV-VR538_P64L_3P
  misc_feature (36)..(37): Diribonucleotides
ggagctgtcgttcactcgatactggctggtgaaggug

SEQ ID NO: 34 - DNA, 37 bp, Human cytomegalovirus
  misc_feature (1)..(37): Ligation probe name CMV-VR538_P64L_5P
  misc_feature (1)..(1): 5′ phosphorylated
gggtgctcgctgtactgaggagatcggaagagcacac

SEQ ID NO: 35 - DNA, 37 bp, Human cytomegalovirus
  misc_feature (1)..(37): Ligation probe name CMV-VR538_P61L_3P
  misc_feature (36)..(37): Diribonucleotides
ggagctgtcgttcactcgtatggcttgtacctccagg

SEQ ID NO: 36 - DNA, 37 bp, Human cytomegalovirus
  misc_feature (1)..(37): Ligation probe name CMV-VR538_P61L_5P
  misc_feature (1)..(1): 5′ phosphorylated
aagatctgctgcccgttcatagatcggaagagcacac

SEQ ID NO: 37 - DNA, 37 bp, Human cytomegalovirus
  misc_feature (1)..(37): Ligation probe name CMV-VR538_P4L_3P
  misc_feature (36)..(37): Diribonucleotides
ggagctgtcgttcactccgagcagaccaaggacgtug

SEQ ID NO: 38 - DNA, 37 bp, Human cytomegalovirus
  misc_feature (1)..(37): Ligation probe name CMV-VR538_P4L_5P
  misc_feature (1)..(1): 5′ phosphorylated
atgagttgctctaagttcatagatcggaagagcacac

SEQ ID NO: 39 - DNA, 37 bp, Human cytomegalovirus
  misc_feature (1)..(37): Ligation probe name CMV-VR538_P12L_3P
  misc_feature (36)..(37): Diribonucleotides
ggagctgtcgttcactcgtagaaatcatgaagctcac

SEQ ID NO: 40 - DNA, 37 bp, Human cytomegalovirus
  misc_feature (1)..(37): Ligation probe name CMV-VR538_P12L_5P
  misc_feature (1)..(1): 5′ phosphorylated
gataaacgagtccggagccaagatcggaagagcacac

SEQ ID NO: 41 - DNA, 37 bp, Human cytomegalovirus
  misc_feature (1)..(37): Ligation probe name CMV-VR538_P18L_3P
  misc_feature (36)..(37): Diribonucleotides
ggagctgtcgttcactcgtgaatgctgctttgtcccu

SEQ ID NO: 42 - DNA, 37 bp, Human cytomegalovirus
  misc_feature (1)..(37): Ligation probe name CMV-VR538_P18L_5P
  misc_feature (1)..(1): 5′ phosphorylated
tgatatagccttgttactctagatcggaagagcacac

SEQ ID NO: 43 - DNA, 37 bp, Human cytomegalovirus
  misc_feature (1)..(37): Ligation probe name CMV-VR538_P2L_3P
  misc_feature (36)..(37): Diribonucleotides
ggagctgtcgttcactccaaggccgggatctacacag

SEQ ID NO: 44 - DNA, 37 bp, Human cytomegalovirus
  misc_feature (1)..(37): Ligation probe name CMV-VR538_P2L_5P
  misc_feature (1)..(1): 5′ phosphorylated
tcagagtagcctagaaacatagatcggaagagcacac

SEQ ID NO: 45 - DNA, 37 bp, Human cytomegalovirus
  misc_feature (1)..(37): Ligation probe name CMV-VR538_P31L_3P
  misc_feature (36)..(37): Diribonucleotides
ggagctgtcgttcactccggggagtatgacgttttga

SEQ ID NO: 46 - DNA, 37 bp, Human cytomegalovirus
  misc_feature (1)..(37): Ligation probe name CMV-VR538_P31L_5P
  misc_feature (1)..(1): 5′ phosphorylated
ttacagacggagatggcagcagatcggaagagcacac

SEQ ID NO: 47 - DNA, 37 bp, Human cytomegalovirus
  misc_feature (1)..(37): Ligation probe name CMV-VR538_P11L_3P
  misc_feature (36)..(37): Diribonucleotides
ggagctgtcgttcactcgcgcaggtaaacggaatcug

SEQ ID NO: 48 - DNA, 37 bp, Human cytomegalovirus
  misc_feature (1)..(37): Ligation probe name CMV-VR538_P11L_5P
  misc_feature (1)..(1): 5′ phosphorylated
gggaattcaacacaggtaagagatcggaagagcacac

SEQ ID NO: 49 - DNA, 37 bp, Influenza A virus
  misc_feature (1)..(37): Ligation probe name OI_H1N1_P13L_3P
  misc_feature (36)..(37): Diribonucleotides
ggagctgtcgttcactctgggaagtgttgaaccttgc

SEQ ID NO: 50 - DNA, 37 bp, Influenza A virus
  misc_feature (1)..(37): Ligation probe name OI_H1N1_P13L_5P
  misc_feature (1)..(1): 5′ phosphorylated
attagagagcacattctgggagatcggaagagcacac

SEQ ID NO: 51 - DNA, 37 bp, Influenza A virus
  misc_feature (1)..(37): Ligation probe name OI_H1N1_P21L_3P
  misc_feature (36)..(37): Diribonucleotides
ggagctgtcgttcactccacacaagcaggcaggcagg

SEQ ID NO: 52 - DNA, 37 bp, Influenza A virus
  misc_feature (1)..(37): Ligation probe name OI_H1N1_P21L_5P
  misc_feature (1)..(1): 5′ phosphorylated
atttatgtgcaactgatcccagatcggaagagcacac

SEQ ID NO: 53 - DNA, 37 bp, Influenza A virus
  misc_feature (1)..(37): Ligation probe name OI_Pan_Mseg_1_3P
  misc_feature (36)..(37): Diribonucleotides
ggagctgtcgttcactcggtcttgtctttagccatuc

SEQ ID NO: 54 - DNA, 37 bp, Influenza A virus
  misc_feature (1)..(37): Ligation probe name OI_Pan_Mseg_1_5P
  misc_feature (1)..(1): 5′ phosphorylated
catgagagcctcaagatctgagatcggaagagcacac

SEQ ID NO: 55 - DNA, 37 bp, Influenza A virus
  misc_feature (1)..(37): Ligation probe name OI_H1N1_P4L_3P
  misc_feature (36)..(37): Diribonculeotides
ggagctgtcgttcactccatcataatcactgagttug

SEQ ID NO: 56 - DNA, 37 bp, Influenza A virus
  misc_feature (1)..(37): Ligation probe name OI_H1N1_P4L_5P
  misc_feature (1)..(1): 5′ phosphorylated
agttcagtgcacatttggatagatcggaagagcacac

SEQ ID NO: 57 - DNA, 37 bp, Mycobacterium smegmatis
  misc_feature (1)..(37): Ligation probe name MS-14468_P24L_3P
  misc_feature (36)..(37): Diribonucleotides
ggagctgtcgttcactccatctctgccggcgtcctgu

SEQ ID NO: 58 - DNA, 37 bp, Mycobacterium smegmatis
  misc_feature (1)..(37): Ligation probe name MS-14468_P24L_5P
  misc_feature (1)..(1): 5′ phosphorylated
gcatgtcaaacccaggtaagagatcggaagagcacac

SEQ ID NO: 59 - DNA, 37 bp, Mycobacterium smegmatis
  misc_feature (1)..(37): Ligation probe name MS-14468_P34L_3P
  misc_feature (36)..(37): Diribonucleotides
ggagctgtcgttcactctttcatgacgtgacgggcgg

SEQ ID NO: 60 - DNA, 37 bp, Mycobacterium smegmatis
  misc_feature (1)..(37): Ligation probe name MS-14468_P34L_5P
  misc_feature (1)..(1): 5′ phosphorylated
tgtgtacaaggcccgggaacagatcggaagagcacac

SEQ ID NO: 61 - DNA, 37 bp, Mycobacterium smegmatis
  misc_feature (1)..(37): Ligation probe name MS-14468_P8L_3P
  misc_feature (36)..(37): Diribonucleotides
ggagctgtcgttcactccccactgctgcctcccgtag

SEQ ID NO: 62 - DNA, 37 bp, Mycobacterium smegmatis
  misc_feature (1)..(37): Ligation probe name MS-14468_P8L_5P
  misc_feature (1)..(1): 5′ phosphorylated
cagtctgggccgtatctcagagatcggaagagcacac

SEQ ID NO: 63 - DNA, 37 bp, Pseudomonas aeruginosa
  misc_feature (1)..(37): Ligation probe name PA-10145_P28L_3P
  misc_feature (36)..(37): Diribonucleotides
ggagctgtcgttcactcgtgctggtaactaaggacaa

SEQ ID NO: 64 - DNA, 37 bp, Pseudomonas aeruginosa
  misc_feature (1)..(37): Ligation probe name PA-10145_P28L_5P
  misc_feature (1)..(1): 5′ phosphorylated
gggttgcgctcgttacgggaagatcggaagagcacac

SEQ ID NO: 65 - DNA, 37 bp, Pseudomonas aeruginosa
  misc_feature (1)..(37): Ligation probe name PA-10145_P17L_3P
  misc_feature (36)..(37): Diribonucleotides
ggagctgtcgttcactctcaccgctacacaggaaauu

SEQ ID NO: 66 - DNA, 37 bp, Pseudomonas aeruginosa
  misc_feature (1)..(37): Ligation probe name PA-10145_P17L_5P
  misc_feature (1)..(1): 5′ phosphorylated
ccaccaccctctaccgtactagatcggaagagcacac

SEQ ID NO: 67 - DNA, 37 bp, Pseudomonas aeruginosa
  misc_feature (1)..(37): Ligation probe name PA-10145_P11L_3P
  misc_feature (36)..(37): Diribonucleotides
ggagctgtcgttcactcccttcctcccaacttaaagu

SEQ ID NO: 68 - DNA, 37 bp, Pseudomonas aeruginosa
  misc_feature (1)..(37): Ligation probe name PA-10145_P11L_5P
  misc_feature (1)..(1): 5′ phosphorylated
gctttacaatccgaagacctagatcggaagagcacac

SEQ ID NO: 69 - DNA, 37 bp, Staphylococcus aureus
  misc_feature (1)..(37): Ligation probe name SA-25923_P5L_3P
  misc_feature (36)..(37): Diribonucleotides
ggagctgtcgttcactcattccagcttcatgtagtcg

SEQ ID NO: 70 - DNA, 37 bp, Staphylococcus aureus
  misc_feature (1)..(37): Ligation probe name SA-25923_P5L_5P
  misc_feature (1)..(1): 5′ phosphorylated
agttgcagactacaatccgaagatcggaagagcacac

SEQ ID NO: 71 - DNA, 37 bp, Staphylococcus aureus
  misc_feature (1)..(37): Ligation probe name S_aureus_ATCC_25923_3P
  misc_feature (36)..(37): Diribonucleotides
ggagctgtcgttcactctcgctgccctttgtattguc

SEQ ID NO: 72 - DNA, 37 bp, Staphylococcus aureus
  misc_feature (1)..(37): Ligation probe name S_aureus_ATCC_25923_5P
  misc_feature (1)..(1): 5′ phosphorylated
cattgtagcacgtgtgtagcagatcggaagagcacac

SEQ ID NO: 73 - DNA, 37 bp, Staphylococcus aureus
  misc_feature (1)..(37): Ligation probe name S_aureus_ATCC_25923_3P
  misc_feature (36)..(37): Diribonucleotides
ggagctgtcgttcactccttcctccggtttgtcaccg

SEQ ID NO: 74 - DNA, 37 bp, Staphylococcus aureus
  misc_feature (1)..(37): Ligation probe name S_aureus_ATCC_25923_5P
  misc_feature (1)..(1): 5′ phosphorylated
gcagtcaacttagagtgcccagatcggaagagcacac

SEQ ID NO: 75 - DNA, 37 bp, Zika virus
  misc_feature (1)..(37): Ligation probe name Z_P41L_3P
  misc_feature (36)..(37): Diribonucleotides
ggagctgtcgttcactccaggaggaaggatgtatgca

SEQ ID NO: 76 - DNA, 37 bp, Zika virus
  misc_feature (1)..(37): Ligation probe name Z_P41L_5P
  misc_feature (1)..(1): 5′ phosphorylated
gatgacactgctggctgggaagatcggaagagcacac

SEQ ID NO: 77 - DNA, 37 bp, Zika virus
  misc_feature (1)..(37): Ligation probe name Z_P81L_3P
  misc_feature (36)..(37): Diribonucleotides
ggagctgtcgttcactcccctgggagagaaatggaag

SEQ ID NO: 78 - DNA, 37 bp, Zika virus
  misc_feature (1)..(37): Ligation probe name Z_P81L_5P
  misc_feature (1)..(1): 5′ phosphorylated
gcccgcttgaaccagatgtcagatcggaagagcacac

SEQ ID NO: 79 - DNA, 37 bp, Zika virus
  misc_feature (1)..(37): Ligation probe name Z_P217L_3P
  misc_feature (36)..(37): Diribonucleotides
ggagctgtcgttcactcttgggagacacagcctggga

SEQ ID NO: 80 - DNA, 37 bp, Zika virus
  misc_feature (1)..(37): Ligation probe name Z_P217L_5P
  misc_feature (1)..(1): 5′ phosphorylated
ctttggatcagttggaggcgagatcggaagagcacac

SEQ ID NO: 81 - DNA, 20 bp, Candida albicans
  misc_feature (1)..(20): Capture probe name CA-18804__P7C_Cap
gcgttcaaagattcgatgat

SEQ ID NO: 82 - DNA, 20 bp, Candida albicans
  misc_feature (1)..(20): Capture probe name C_albicans_ATCC_18804_ITS_Cap
gcgttcaaagattcgatgat

SEQ ID NO: 83 - DNA, 20 bp, Cryptococcus neoformans
  misc_feature (1)..(20): Capture probe name CN-32045__P6C_Cap
gactcgatgattcactgaat

SEQ ID NO: 84 - DNA, 20 bp, Cryptococcus neoformans
  misc_feature (1)..(20): Capture probe name CN-32045__P1_2C_Cap
caccgatcagaactgatgtt

SEQ ID NO: 85 - DNA, 20 bp, Cryptococcus neoformans
  misc_feature (1)..(20): Capture probe name CN-32045_18S_P3_4C_Cap
cccagtcagagattgacgtg

SEQ ID NO: 86 - DNA, 20 bp, Cryptococcus neoformans
  misc_feature (1)..(20): Capture probe name C_neoformans_ATCC_32045_ITS_Cap
gactcgatgattcactgaat

SEQ ID NO: 87 - DNA, 20 bp, Haemophilus influenzae
  misc_feature (1)..(20): Capture probe name HI-10211_P34C_Cap
catctctacgcatttcaccg

SEQ ID NO: 88 - DNA, 20 bp, Haemophilus influenzae
  misc_feature (1)..(20): Capture probe name HI-10211_P1C_Cap
cgttcgacttgcatgtgtta

SEQ ID NO: 89 - DNA, 20 bp, Human cytomegalovirus
  misc_feature (1)..(20): Capture probe name CMV-VR538_P60C_Cap
tcaggcccgggatgctcttg

SEQ ID NO: 90 - DNA, 20 bp, Human cytomegalovirus
  misc_feature (1)..(20): Capture probe name CMV-VR538_P16C_Cap
aggtccagctgcagataccc

SEQ ID NO: 91 - DNA, 20 bp, Human cytomegalovirus
  misc_feature (1)..(20): Capture probe name CMV-VR538_P26C_Cap
cttcctcccgcgacaacagc

SEQ ID NO: 92 - DNA, 20 bp, Human cytomegalovirus
  misc_feature (1)..(20): Capture probe name CMV-VR538_P7C_Cap
gcaccttctcagaaatcgaa

SEQ ID NO: 93 - DNA, 20 bp, Influenza A virus
  misc_feature (1)..(20): Capture probe name OI_H1N1_P11C_Cap
gtggcatcattcaggttgga

SEQ ID NO: 94 - DNA, 26 bp, Influenza A virus
  misc_feature (1)..(26): Capure probe name OI_Mseg_Pan__Cap
gcgatctcggctttgagggggcctga

SEQ ID NO: 95 - DNA, 20 bp, Mycobacterium smegmatis
  misc_feature (1)..(20): Capture probe name MS-14468_P25C_Cap
acaggccacaagggaaccga

SEQ ID NO: 96 - DNA, 20 bp, Mycobacterium smegmatis
  misc_feature (1)..(20): Capture probe name MS-14468_P9C_Cap
gcgcccattgtgcaatattc

SEQ ID NO: 97 - DNA, 20 bp, Pseudomonas aeruginosa
  misc_feature (1)..(20): Capture probe name PA-10145_P20C_Cap
tatctaatcctgtttgctcc

SEQ ID NO: 98 - DNA, 20 bp, Zika virus
  misc_feature (1)..(20): Capture probe name Z_P44C_Cap
gtgttgaagggctgggatta

SEQ ID NO: 99 - DNA, 20 bp, Zika virus
  misc_feature (1)..(20): Capture probe name Z_P79C_Cap
ccgccgcgccctcaaggacg

SEQ ID NO: 100 - DNA, 20 bp, Zika virus
  misc_feature (1)..(20): Capture probe name Z_P218C_Cap
gtgccaagagaatggcagtc

SEQ ID NO: 101 - DNA, 37 bp, Human coronavirus 229E
  misc_feature (1)..(37): 3P Probe name 3P_KY369909.2_12844-12883 (detection)
  misc_feature (36)..(37): Diribonucleotides
ggagctgtcgttcactcgtcaaaccaattcttcatuu

SEQ ID NO: 102 - DNA, 37 bp, Human coronavirus 229E
  misc_feature (1)..(37): 5P Probe name 5P_KY369909.2_12844-12883 (detection)
  misc_feature (1)..(1): 5′ phosphorylated
caaagtaatcagtattacaaagatcggaagagcacac

SEQ ID NO: 103 - DNA, 37 bp, Human coronavirus 229E
  misc_feature (1)..(37): 3P Probe name 3P_KY369909.2_21676-21715 (detection)
  misc_feature (36)..(37): Diribonucleotides
ggagctgtcgttcactccctatattatgagatttaug

SEQ ID NO: 104 - DNA, 37 bp, Human coronavirus 229E
  misc_feature (1)..(37): 5P Probe name 5P_KY369909.2_21676-21715 (detection)
  misc_feature (1)..(1): 5′ phosphorylated
attaaccaaattagccattaagatcggaagagcacac

SEQ ID NO: 105 - DNA, 37 bp, Human coronavirus 229E
  misc_feature (1)..(37): 3P Probe name 3P_KY369909.2_22427-22466 (detection)
  misc_feature (36)..(37): Diribonucleotides
ggagctgtcgttcactcattagcaagtgtaaacgcuu

SEQ ID NO: 106 - DNA, 37 bp, Human coronavirus 229E
  misc_feature (1)..(37): 5P Probe name 5P_KY369909.2_22427-22466 (detection)
  misc_feature (1)..(1): 5′ phosphorylated
tcttgtcaaaagtgagcatcagatcggaagagcacac

SEQ ID NO: 107 - DNA, 37 bp, SARS-CoV-2
  misc_feature (1)..(37): 3P Probe name 3P_CV_NC_045512.2_1188-1627mt (genotyping)
  misc_feature (36)..(37): Diribonucleotides
ggagctgtcgttcactcggtattcggcaagactatgc

SEQ ID NO: 108 - DNA, 37 bp, SARS-CoV-2
  misc_feature (1)..(37): 5P Probe name 5P_CV_NC_045512.2_1188-1627mt (genotyping)
  misc_feature (1)..(1): 5′ phosphorylated
tcaggtcctatttctgaattagatcggaagagcacac

SEQ ID NO: 109 - DNA, 37 bp, SARS-CoV-2; identical to SEQ ID NO: 107
  misc_feature (1)..(37): 3P Probe name 3P_CV_NC_045512.2_1188-1627wt_G-A (genotyping)
  misc_feature (36)..(37): Diribonucleotides

SEQ ID NO: 110 - DNA, 37 bp, SARS-CoV-2
  misc_feature (1)..(37): 5P Probe name 5P_CV_NC_045512.2_1188-1627wt_G-A (genotyping)
  misc_feature (1)..(1): 5′ phosphorylated
tcaggtcctacttctgaattagatcggaagagcacac

SEQ ID NO: 111 - DNA, 37 bp, SARS-CoV-2
  misc_feature (1)..(37): 3P Probe name 3P_CV_NC_045512.2_14199-14638mt (genotyping)
  misc_feature (36)..(37): Diribonucleotides
ggagctgtcgttcactcatttttctcactagtggtcc

SEQ ID NO: 112 - DNA, 37 bp, SARS-CoV-2
  misc_feature (1)..(37): 5P Probe name 5P_CV_NC_045512.2_14199-14638mt (genotyping)
  misc_feature (1)..(1): 5′ phosphorylated
aaaacttgtaagtgggaacaagatcggaagagcacac

SEQ ID NO: 113 - DNA, 37 bp, SARS-CoV-2; identical to SEQ ID NO: 111
  misc_feature (1)..(37): 3P Probe name 3P_CV_NC_045512.2_14199-14638wt_C-T (genotyping)
  misc_feature (36)..(37): Diribonucleotides

SEQ ID NO: 114 - DNA, 37 bp, SARS-CoV-2
  misc_feature (1)..(37): 5P Probe name 5P_CV_NC_045512.2_14199-14638wt_C-T (genotyping)
  misc_feature (1)..(1): 5′ phosphorylated
aaaacttgtaggtgggaacaagatcggaagagcacac

SEQ ID NO: 115 - DNA, 37 bp, SARS-CoV-2
  misc_feature (1)..(37): 3P Probe name 3P_CV_NC_045512.2_14596-15035mt (genotyping)
  misc_feature (36)..(37): Diribonucleotides
ggagctgtcgttcactcatcacacattgttggtagau

SEQ ID NO: 116 - DNA, 37 bp, SARS-CoV-2
  misc_feature (1)..(37): 5P Probe name 5P_CV_NC_045512.2_14596-15035mt (genotyping)
  misc_feature (1)..(1): 5′ phosphorylated
tataacgataatagtcataaagatcggaagagcacac

SEQ ID NO: 117 - DNA, 37 bp, SARS-CoV-2; identical to SEQ ID NO: 115
  misc_feature (1)..(37): 3P Probe name 3P_CV_NC_045512.2_14596-15035wt_C-T (genotyping)
  misc_feature (36)..(37): Diribonucleotides

SEQ ID NO: 118 - DNA, 37 bp, SARS-CoV-2
  misc_feature (1)..(37): 5P Probe name 5P_CV_NC_045512.2_14596-15035wt_C-T (genotyping)
  misc_feature (1)..(1): 5′ phosphorylated
tataacgatagtagtcataaagatcggaagagcacac

SEQ ID NO: 119 - DNA, 37 bp, SARS-CoV-2
  misc_feature (1)..(37): 3P Probe name 3P_CV_NC_045512.2_17538-17977mt (genotyping)
  misc_feature (36)..(37): Diribonucleotides
ggagctgtcgttcactcggtgaaataaagacagctuu

SEQ ID NO: 120 - DNA, 37 bp, SARS-CoV-2
  misc_feature (1)..(37): 5P Probe name 5P_CV_NC_045512.2_17538-17977mt (genotyping)
  misc_feature (1)..(1): 5′ phosphorylated
tctccaagcaaggttacgtgagatcggaagagcacac

SEQ ID NO: 121 - DNA, 37 bp, SARS-CoV-2; identical to SEQ ID NO: 119
  misc_feature (1)..(37): 3P Probe name 3P_CV_NC_045512.2_17538-17977wt_C-T (genotyping)
  misc_feature (36)..(37): Diribonucleotides

SEQ ID NO: 122 - DNA, 37 bp, SARS-CoV-2
  misc_feature (1)..(37): 5P Probe name 5P_CV_NC_045512.2_17538-17977wt_C-T (genotyping)
  misc_feature (1)..(1): 5′ phosphorylated
tctccaagcagggttacgtgagatcggaagagcacac

SEQ ID NO: 123 - DNA, 37 bp, SARS-CoV-2
  misc_feature (1)..(37): 3P Probe name 3P_CV_NC_045512.2_17649-18088mt (genotyping)
  misc_feature (36)..(37): Diribonucleotides
ggagctgtcgttcactctcagtggtttgagtgaatau

SEQ ID NO: 124 - DNA, 37 bp, SARS-CoV-2
  misc_feature (1)..(37): 5P Probe name 5P_CV_NC_045512.2_17649-18088mt (genotyping)
  misc_feature (1)..(1): 5′ phosphorylated
gacatagtcacattctgagcagatcggaagagcacac

SEQ ID NO: 125 - DNA, 37 bp, SARS-CoV-2; identical to SEQ ID NO: 123
  misc_feature (1)..(37): 3P Probe name 3P_CV_NC_045512.2_17649-18088wt_A-G (genotyping)
  misc_feature (36)..(37): Diribonucleotides

SEQ ID NO: 126 - DNA, 37 bp, SARS-CoV-2
  misc_feature (1)..(37): 5P Probe name 5P_CV_NC_045512.2_17649-18088wt_A-G (genotyping)
  misc_feature (1)..(1): 5′ phosphorylated
gacatagtcatattctgagcagatcggaagagcacac

SEQ ID NO: 127 - DNA, 37 bp, SARS-CoV-2
  misc_feature (1)..(37): 3P Probe name 3P_CV_NC_045512.2_17851-18290mt (genotyping)
  misc_feature (36)..(37): Diribonucleotides
ggagctgtcgttcactccccagtgattaccttactac

SEQ ID NO: 128 - DNA, 37 bp, SARS-CoV-2
  misc_feature (1)..(37): 5P Probe name 5P_CV_NC_045512.2_17851-18290mt (genotyping)
  misc_feature (1)..(1): 5′ phosphorylated
aatctttaaaaagtcctgttagatcggaagagcacac

SEQ ID NO: 129 - DNA, 37 bp, SARS-CoV-2; identical to SEQ ID NO: 127
  misc_feature (1)..(37): 3P Probe name 3P_CV_NC_045512.2_17851-18290wt_C-T (genotyping)
  misc_feature (36)..(37): Diribonucleotides

SEQ ID NO: 130 - DNA, 37 bp, SARS-CoV-2
  misc_feature (1)..(37): 5P Probe name 5P_CV_NC_045512.2_17851-18290wt_C-T (genotyping)
  misc_feature (1)..(1): 5′ phosphorylated
aatctttaaagagtcctgttagatcggaagagcacac

SEQ ID NO: 131 - DNA, 37 bp, SARS-CoV-2
  misc_feature (1)..(37): 3P Probe name 3P_CV_NC_045512.2_23194-23633mt (genotyping)
  misc_feature (36)..(37): Diribonucleotides
ggagctgtcgttcactcatagcaacagggacttctgu

SEQ ID NO: 132 - DNA, 37 bp, SARS-CoV-2
  misc_feature (1)..(37): 5P Probe name 5P_CV_NC_045512.2_23194-23633mt (genotyping)
  misc_feature (1)..(1): 5′ phosphorylated
gcagttaacaccctgataaaagatcggaagagcacac

SEQ ID NO: 133 - DNA, 37 bp, SARS-CoV-2; identical to SEQ ID NO: 131
  misc_feature (1)..(37): 3P Probe name 3P_CV_NC_045512.2_23194-23633wt_A-G (genotyping)
  misc_feature (36)..(37): Diribonucleotides

SEQ ID NO: 134 - DNA, 37 bp, SARS-CoV-2
  misc_feature (1)..(37): 5P Probe name 5P_CV_NC_045512.2_23194-23633wt_A-G (genotyping)
  misc_feature (1)..(1): 5′ phosphorylated
gcagttaacatcctgataaaagatcggaagagcacac

SEQ ID NO: 135 - DNA, 37 bp, SARS-CoV-2
  misc_feature (1)..(37): 3P Probe name 3P_CV_NC_045512.2_27935-28374mt (genotyping)
  misc_feature (36)..(37): Diribonucleotides
ggagctgtcgttcactcttaggttcctggcaattaau

SEQ ID NO: 136 - DNA, 37 bp, SARS-CoV-2
  misc_feature (1)..(37): 5P Probe name 5P_CV_NC_045512.2_27935-28374mt (genotyping)
  misc_feature (1)..(1): 5′ phosphorylated
tgtaaaaggtgaacaggaaaagatcggaagagcacac

SEQ ID NO: 137 - DNA, 37 bp, SARS-CoV-2; identical to SEQ ID NO: 135
  misc_feature (1)..(37): 3P Probe name 3P_CV_NC_045512.2_27935-28374wt_T-C (genotyping)
  misc_feature (36)..(37): Diribonucleotides

SEQ ID NO: 138 - DNA, 37 bp, SARS-CoV-2
  misc_feature (1)..(37): 5P Probe name 5P_CV_NC_045512.2_27935-28374wt_T-C (genotyping)
  misc_feature (1)..(1): 5′ phosphorylated
tgtaaaaggtaaacaggaaaagatcggaagagcacac

SEQ ID NO: 139 - DNA, 37 bp, SARS-CoV-2
  misc_feature (1)..(37): 3P Probe name 3P_CV_NC_045512.2_2828-3267mt (genotyping)
  misc_feature (36)..(37): Diribonucleotides
ggagctgtcgttcactcaccttcttcttcatcctcau

SEQ ID NO: 140 - DNA, 37 bp, SARS-CoV-2
  misc_feature (1)..(37): 5P Probe name 5P_CV_NC_045512.2_2828-3267mt (genotyping)
  misc_feature (1)..(1): 5′ phosphorylated
ctggagggtaaaaagaacaaagatcggaagagcacac

SEQ ID NO: 141 - DNA, 37 bp, SARS-CoV-2; identical to SEQ ID NO: 139
  misc_feature (1)..(37): 3P Probe name 3P_CV_NC_045512.2_2828-3267wt_C-T (genotyping)
  misc_feature (36)..(37): Diribonucleotides

SEQ ID NO: 142 - DNA, 37 bp, SARS-CoV-2
  misc_feature (1)..(37): 5P Probe name 5P_CV_NC_045512.2_2828-3267wt_C-T (genotyping)
  misc_feature (1)..(1): 5′ phosphorylated
ctggagggtagaaagaacaaagatcggaagagcacac

SEQ ID NO: 143 - DNA, 37 bp, SARS-CoV-2
  misc_feature (1)..(37): 3P Probe name 3P_CV_NC_045512.2_28479-28918mt (genotyping)
  misc_feature (36)..(37): Diribonucleotides
ggagctgtcgttcactcgggtgccaatgtgatcttuu

SEQ ID NO: 144 - DNA, 37 bp, SARS-CoV-2
  misc_feature (1)..(37): 5P Probe name 5P_CV_NC_045512.2_28479-28918mt (genotyping)
  misc_feature (1)..(1): 5′ phosphorylated
ggtgtattcagggctccctcagatcggaagagcacac

SEQ ID NO: 145 - DNA, 37 bp, SARS-CoV-2; identical to SEQ ID NO: 143
  misc_feature (1)..(37): 3P Probe name 3P_CV_NC_045512.2_28479-28918wt_T-C (genotyping)
  misc_feature (36)..(37): Diribonucleotides

SEQ ID NO: 146 - DNA, 37 bp, SARS-CoV-2
  misc_feature (1)..(37): 5P Probe name 5P_CV_NC_045512.2_28479-28918wt_T-C (genotyping)
  misc_feature (1)..(1): 5′ phosphorylated
ggtgtattcaaggctccctcagatcggaagagcacac

SEQ ID NO: 147 - DNA, 37 bp, SARS-CoV-2
  misc_feature (1)..(37): 3P Probe name 3P_CV_NC_045512.2_28672-29111mt (genotyping)
  misc_feature (36)..(37): Diribonucleotides
ggagctgtcgttcactcttgccagccattctagcagg

SEQ ID NO: 148 - DNA, 37 bp, SARS-CoV-2
  misc_feature (1)..(37): 5P Probe name 5P_CV_NC_045512.2_28672-29111mt (genotyping)
  misc_feature (1)..(1): 5′ phosphorylated
agaagttcccttactgctgcagatcggaagagcacac

SEQ ID NO: 149 - DNA, 39 bp, SARS-CoV-2
  misc_feature (1)..(37): 3P Probe name 3P_CV_NC_045512.2_28672-29111wt_G-A (genotyping)
  misc_feature (36)..(37): Diribonucleotides
ggagctgtcgttcactcttgccagccattctagcargrg

SEQ ID NO: 150 - DNA, 37 bp, SARS-CoV-2
  misc_feature (1)..(37): 5P Probe name 5P_CV_NC_045512.2_28672-29111wt_G-A (genotyping)
  misc_feature (1)..(1): 5′ phosphorylated
agaagttcccctactgctgcagatcggaagagcacac

SEQ ID NO: 151 - DNA, 37 bp, SARS-CoV-2
  misc_feature (1)..(37): 3P Probe name 3P_CV_NC_045512.2_28886-29325mt (genotyping)
  misc_feature (36)..(37): Diribonucleotides
ggagctgtcgttcactctccttgggtttgttctggac

SEQ ID NO: 152 - DNA, 37 bp, SARS-CoV-2
  misc_feature (1)..(37): 5P Probe name 5P_CV_NC_045512.2_28886-29325mt (genotyping)
  misc_feature (1)..(1): 5′ phosphorylated
cacgtctgccaaaagcttgtagatcggaagagcacac

SEQ ID NO: 153 - DNA, 37 bp, SARS-CoV-2; identical to SEQ ID NO: 151
  misc_feature (1)..(37): 3P Probe name 3P_CV_NC_045512.2_28886-29325wt_C-T (genotyping)
  misc_feature (36)..(37): Diribonucleotides

SEQ ID NO: 154 - DNA, 37 bp, SARS-CoV-2
  misc_feature (1)..(37): 5P Probe name 5P_CV_NC_045512.2_28886-29325wt_C-T (genotyping)
  misc_feature (1)..(1): 5′ phosphorylated
cacgtctgccgaaagcttgtagatcggaagagcacac

SEQ ID NO: 155 - DNA, 37 bp, SARS-CoV-2
  misc_feature (1)..(37): 3P Probe name 3P_CV_NC_045512.2_29533-29972mt (genotyping)
  misc_feature (36)..(37): Diribonucleotides
ggagctgtcgttcactcattgttcactgtacactcga

SEQ ID NO: 156 - DNA, 37 bp, SARS-CoV-2
  misc_feature (1)..(37): 5P Probe name 5P_CV_NC_045512.2_29533-29972mt (genotyping)
  misc_feature (1)..(1): 5′ phosphorylated
tcgtactccgagtggcctcgagatcggaagagcacac

SEQ ID NO: 157 - DNA, 37 bp, SARS-CoV-2; identical to SEQ ID NO: 155
  misc_feature (1)..(37): 3P Probe name 3P_CV_NC_045512.2_29533-29972wt_G-T (genotyping)
  misc_feature (36)..(37): Diribonucleotides

SEQ ID NO: 158 - DNA, 37 bp, SARS-CoV-2
  misc_feature (1)..(37): 5P Probe name 5P_CV_NC_045512.2_29533-29972wt_G-T (genotyping)
  misc_feature (1)..(1): 5′ phosphorylated
tcgtactccgcgtggcctcgagatcggaagagcacac

SEQ ID NO: 159 - DNA, 37 bp, SARS-CoV-2
  misc_feature (1)..(37): 3P Probe name 3P_CV_NC_045512.2_305-744mt (genotyping)
  misc_feature (36)..(37): Diribonucleotides
ggagctgtcgttcactcttcgagttctgctaccagcu

SEQ ID NO: 160 - DNA, 37 bp, SARS-CoV-2
  misc_feature (1)..(37): 5P Probe name 5P_CV_NC_045512.2_305-744mt (genotyping)
  misc_feature (1)..(1): 5′ phosphorylated
caaccataacgtgaccatgaagatcggaagagcacac

SEQ ID NO: 161 - DNA, 37 bp, SARS-CoV-2; identical to SEQ ID NO: 159
  misc_feature (1)..(37): 3P Probe name 3P_CV_NC_045512.2_305-744wt_T-C (genotyping)
  misc_feature (36)..(37): Diribonucleotides

SEQ ID NO: 162 - DNA, 37 bp, SARS-CoV-2
  misc_feature (1)..(37): 5P Probe name 5P_CV_NC_045512.2_305-744wt_T-C (genotyping)
  misc_feature (1)..(1): 5′ Phosphorylated
caaccataacatgaccatgaagatcggaagagcacac

SEQ ID NO: 163 - DNA, 37 bp, SARS-CoV-2
  misc_feature (1)..(37): 3P Probe name 3P_CV_NC_045512.2_32-471mt (genotyping)
  misc_feature (1)..(1): Diribonucleotides
ggagctgtcgttcactcctccatcttacctttcgguc

SEQ ID NO: 164 - DNA, 37 bp, SARS-CoV-2
  misc_feature (1)..(37): 5P Probe name 5P_CV_NC_045512.2_32-471mt (genotyping)
  misc_feature (1)..(1): 5′ phosphorylated
acacccggacaaaacctagaagatcggaagagcacac

SEQ ID NO: 165 - DNA, 37 bp, SARS-CoV-2; identical to SEQ ID NO: 163
  misc_feature (1)..(37): 3P Probe name 3P_CV_NC_045512.2_32-471wt_C-T (genotyping)
  misc_feature (36)..(37): Diribonucleotides

SEQ ID NO: 166 - DNA, 37 bp, SARS-CoV-2
  misc_feature (1)..(37): 5P Probe name 5P_CV_NC_045512.2_32-471wt_C-T (genotyping)
  misc_feature (1)..(1): 5′ phosphorylated
acacccggacgaaacctagaagatcggaagagcacac

SEQ ID NO: 167 - DNA, 37 bp, SARS-CoV-2
  misc_feature (1)..(37): 3P Probe name 3P_CV_NC_045512.2_8573-9012mt (genotyping)
  misc_feature (36)..(37): diribonucleotides
ggagctgtcgttcactctttgtcattagtataactac

SEQ ID NO: 168 - DNA, 37 bp, SARS-CoV-2
  misc_feature (1)..(37): 5P Probe name 5P_CV_NC_045512.2_8573-9012mt (genotyping)
  misc_feature (1)..(1): 5′ phosphorylated
caccacgctgactaaaccatagatcggaagagcacac

SEQ ID NO: 169 - DNA, 37 bp, SARS-CoV-2; identical to SEQ ID NO: 167
  misc_feature (1)..(37): 3P Probe name 3P_CV_NC_045512.2_8573-9012wt_C-T (genotyping)
  misc_feature (36)..(37): Diribonucleotides

SEQ ID NO: 170 - DNA, 37 bp, SARS-CoV-2
  misc_feature (1)..(37): 5P Probe name 5P_CV_NC_045512.2_8573-9012wt_C-T (genotyping)
  misc_feature (1)..(1): 5′ phosphorylated
caccacgctggctaaaccatagatcggaagagcacac

SEQ ID NO: 171 - DNA, 37 bp, SARS-CoV-2
  misc_feature (1)..(37): 3P Probe name 3P_hCoV-19|EPI_ISL_423027_26475-26514 (detection)
  misc_feature (36)..(37): Diribonucleotides
ggagctgtcgttcactccagaagatcaggaactctag

SEQ ID NO: 172 - DNA, 37 bp, SARS-CoV-2
  misc_feature (1)..(37): 5P Probe name 5P_hCoV-19|EPI_ISL_423027_26475-26514 (detection)
  misc_feature (1)..(1): 5′ phosphorylated
aagaattcagatttttaacaagatcggaagagcacac

SEQ ID NO: 173 - DNA, 37 bp, SARS-CoV-2
  misc_feature (1)..(37): 3P Probe name 3P_hCoV-19|EPI_ISL_423027_27234-27273 (detection)
  misc_feature (36)..(37): Diribonucleotides
ggagctgtcgttcactcctgaaagtcaacgagatgaa

SEQ ID NO: 174 - DNA, 37 bp, SARS-CoV-2
  misc_feature (1)..(37): 5P Probe name 5P_hCoV-19|EPI_ISL_423027_27234-27273 (detection)
  misc_feature (1)..(1): 5′ phosphorylated
acatctgttgtcacttactgagatcggaagagcacac

SEQ ID NO: 175 - DNA, 37 bp, SARS-CoV-2
  misc_feature (1)..(37): 3P Probe name 3P_hCoV-19|EPI_ISL_423027_27605-27644 (detection)
  misc_feature (36)..(37): Diribonucleotides
ggagctgtcgttcactcggacaagcaaaagcaaatug

SEQ ID NO: 176 - DNA, 37 bp, SARS-CoV-2
  misc_feature (1)..(37): 5P Probe name 5P_hCoV-19|EPI_ISL_423027_27605-27644 (detection)
  misc_feature (1)..(1): 5′ phosphorylated
agtgctaaagcaagtcagtgagatcggaagagcacac

SEQ ID NO: 177 - DNA, 39 bp, SARS-CoV-2
  misc_feature (1)..(37): 3P Probe name 3P_hCoV-19|EPI_ISL_423027_28436-28475 (detection)
  misc_feature (36)..(37): Diribonucleotides
ggagctgtcgttcactcttagtttgttcgtttagarurg

SEQ ID NO: 178 - DNA, 37 bp, SARS-CoV-2
  misc_feature (1)..(37): 5P Probe name 5P_hCoV-19|EPI_ISL_423027_28436-28475 (detection)
  misc_feature (1)..(1): 5′ phosphorylated
aaatctaaaacaacacgaacagatcggaagagcacac

SEQ ID NO: 179 - DNA, 37 bp, SARS-CoV-2
  misc_feature (1)..(37): 3P Probe name 3P_hCoV-19|EPI_ISL_423027_28704-28743 (detection)
  misc_feature (36)..(37): Diribonucleotides
ggagctgtcgttcactctcttcggtagtagccaatuu

SEQ ID NO: 180 - DNA, 37 bp, SARS-CoV-2
  misc_feature (1)..(37): 5P Probe name 5P_hCoV-19|EPI_ISL_423027_28704-28743 (detection)
  misc_feature (1)..(1): 5′ phosphorylated
ggtcatctggactgctattgagatcggaagagcacac

SEQ ID NO: 181 - DNA, 37 bp, NL63
  misc_feature (1)..(37): 3P Probe name 3P_MG428704.1_15317-15356 (detection)
  misc_feature (36)..(37): diribonucleotides
ggagctgtcgttcactctacatgatcatatgcgcauu

SEQ ID NO: 182 - DNA, 37 bp, NL63
  misc_feature (1)..(37): 5P Probe name 5P_MG428704.1_15317-15356 (detection)
  misc_feature (1)..(1): 5′ phosphorylated
tagtgcacaacataggcttaagatcggaagagcacac

SEQ ID NO: 183 - DNA, 37 bp, NL63; identical to SEQ ID NO: 181
  misc_feature (1)..(37): 3P Probe name 3P_MG428704.1_22316-22355 (detection)
  misc_feature (36)..(37): Diribonucleotides

SEQ ID NO: 184 - DNA, 37 bp, NL63; identical to SEQ ID NO: 182
  misc_feature (1)..(37): 5P Probe name 5P_MG428704.1_15317-15356 (detection)
  misc_feature (1)..(1): 5′ phosphorylated

SEQ ID NO: 185 - DNA, 37 bp, NL63
  misc_feature (1)..(37): 3P Probe name 3P_MG428704.1_22316-22355 (detection)
  misc_feature (36)..(37): Diribonucleotides
ggagctgtcgttcactcactacctaactcaatttggu

SEQ ID NO: 186 - DNA, 37 bp, NL63
  misc_feature (1)..(37): 5P Probe name 5P_MG428704.1_9630-9669 (detection)
  misc_feature (1)..(1): 5′ phosphorylated
gtaaataacaaaactcaacaagatcggaagagcacac

SEQ ID NO: 187 - DNA, 37 bp, SARS-CoV
  misc_feature (1)..(37): 3P Probe name 3P_MK062184.1_1570-1609 (detection)
  misc_feature (36)..(37): Diribonucleotides
ggagctgtcgttcactcctcattcaaggtctccacau

SEQ ID NO: 188 - DNA, 37 bp, SARS-CoV
  misc_feature (1)..(37): 5P Probe name 5P_MK062184.1_1570-1609 (detection)
  misc_feature (1)..(1): 5′ phosphorylated
tgtcaccagtaatgccagtaagatcggaagagcacac

SEQ ID NO: 189 - DNA, 37 bp, SARS-CoV
  misc_feature (1)..(37): 3P Probe name 3P_MK062184.1_20150-20189 (detection)
  misc_feature (36)..(37): Diribonucleotides
ggagctgtcgttcactcaagtcagtttccatttgtga

SEQ ID NO: 190 - DNA, 37 bp, SARS-CoV
  misc_feature (1)..(37): 5P Probe name 5P_MK062184.1_20150-20189 (detection)
  misc_feature (1)..(1): 5′ phosphorylated
tctgggcttaaaatcctctaagatcggaagagcacac

SEQ ID NO: 191 - DNA, 37 bp, SARS-CoV
  misc_feature (1)..(37): 3P Probe name 3P_MK062184.1_20306-20345 (detection)
  misc_feature (36)..(37): Diribonucleotides
ggagctgtcgttcactcaatttaagtggtgaatctug

SEQ ID NO: 192 - DNA, 37 bp, SARS-CoV
  misc_feature (1)..(37): 5P Probe name 5P_MK062184.1_20306-20345 (detection)
  misc_feature (1)..(1): 5′ phosphorylated
tgagcgcttggctaagcctaagatcggaagagcacac

SEQ ID NO: 193 - DNA, 37 bp, HKU1
  misc_feature (1)..(37): 3P Probe name 3P_MK167038.1_1563-1602 (detection)
  misc_feature (36)..(37): Diribonucleotides
ggagctgtcgttcactccctgaataaaccaaatcauc

SEQ ID NO: 194 - DNA, 37 bp, HKU1
  misc_feature (1)..(37): 5P Probe name 5P_MK167038.1_1563-1602 (detection)
  misc_feature (1)..(1): 5′ phosphorylated
ataagacttgactgaagattagatcggaagagcacac

SEQ ID NO: 195 - DNA, 37 bp, HKU1
  misc_feature (1)..(37): 3P Probe name 3P_MK167038.1_5955-5994 (detection)
  misc_feature (36)..(37): Diribonucleotides
ggagctgtcgttcactcctacaagcatcataatgtug

SEQ ID NO: 196 - DNA, 37 bp, HKU1
  misc_feature (1)..(37): 5P Probe name 5P_MK167038.1_5955-5994 (detection)
  misc_feature (1)..(1): 5′ phosphorylated
gtaaggtgaaccacatttcaagatcggaagagcacac

SEQ ID NO: 197 - DNA, 37 bp, HKU1
  misc_feature (1)..(37): 3P Probe name 3P_MK167038.1_9770-9809 (detection)
  misc_feature (36)..(37): Diribonucleotides
ggagctgtcgttcactctccattgcaaatgcataauu

SEQ ID NO: 198 - DNA, 37 bp, HKU1
  misc_feature (1)..(37): 5P Probe name 5P_MK167038.1_9770-9809 (detection)
  misc_feature (1)..(1): 5′ phosphorylated
acactaatttcagaaggaaaagatcggaagagcacac

SEQ ID NO: 199 - DNA, 37 bp, MERS-CoV
  misc_feature (1)..(37): 3P Probe name 3P_MK462255.1_12626-12665 (detection)
  misc_feature (36)..(37): Diribonucleotides
ggagctgtcgttcactcattacagttagtttgctcuu

SEQ ID NO: 200 - DNA, 37 bp, MERS-CoV
  misc_feature (1)..(37): 5P Probe name 5P_MK462255.1_12626-12665 (detection)
  misc_feature (1)..(1): 5′ phosphorylated
gacctgcagacacaaccatgagatcggaagagcacac

SEQ ID NO: 201 - DNA, 37 bp, MERS-CoV
  misc_feature (1)..(37): 3P Probe name 3P_MK462255.1_25316-25355 (detection)
  misc_feature (36)..(37): Diribonucleotides
ggagctgtcgttcactcaccagtgcagcacagtatga

SEQ ID NO: 202 - DNA, 37 bp, MERS-CoV
  misc_feature (1)..(37): 5P Probe name 5P_MK462255.1_25316-25355 (detection)
  misc_feature (1)..(1): 5′ phosphorylated
agaagacgcatagagctaagagatcggaagagcacac

SEQ ID NO: 203 - DNA, 37 bp, MERS-CoV
  misc_feature (1)..(37): 3P Probe name 3P_MK462255.1_6234-6273 (detection)
  misc_feature (36)..(37): Diribonucleotides
ggagctgtcgttcactctatgtagatcttctttagac

SEQ ID NO: 204 - DNA, 37 bp, MERS-CoV
  misc_feature (1)..(37): 5P Probe name 5P_MK462255.1_6234-6273 (detection)
  misc_feature (1)..(1): 5′ phosphorylated
agatactcaacaacgggagtagatcggaagagcacac

SEQ ID NO: 205 - DNA, 37 bp, OC43
  misc_feature (1)..(37): 3P Probe name 3P_MN306053.1_2448-2487 (detection)
  misc_feature (36)..(37): Diribonucleotides
ggagctgtcgttcactcaaaacaagaaaggccatcaa

SEQ ID NO: 206 - DNA, 37 bp, OC43
  misc_feature (1)..(37): 5P Probe name 5P_MN306053.1_2448-2487 (detection)
  misc_feature (1)..(1): 5′ phosphorylated
taaaagtaactcttaaagagagatcggaagagcacac

SEQ ID NO: 207 - DNA, 37 bp, OC43
  misc_feature (1)..(37): 3P Probe name 3P_MN306053.1_28004-28043 (detection)
  misc_feature (36)..(37): Diribonucleotides
ggagctgtcgttcactctgattacggcaccaagcaug

SEQ ID NO: 208 - DNA, 37 bp, OC43
  misc_feature (1)..(37): 5P Probe name 5P_MN306053.1_28004-28043 (detection)
  misc_feature (1)..(1): 5′ phosphorylated
actaggaactctacaatatcagatcggaagagcacac

SEQ ID NO: 209 - DNA, 37 bp, OC43
  misc_feature (1)..(37): 3P Probe name 3P_MN306053.1_3885-3924 (detection)
  misc_feature (36)..(37): Diribonucleotides
ggagctgtcgttcactcagtaataaatgcacaaaaca

SEQ ID NO: 210 - DNA, 37 bp, OC43
  misc_feature (1)..(37): 5P Probe name 5P_MN306053.1_3885-3924 (detection)
  misc_feature (1)..(1): 5′ phosphorylated
acttatctttaagagcaaagagatcggaagagcacac

SEQ ID NO: 211 - DNA, 37 bp, Influenza A virus; identical to SEQ ID NO: 53
  misc_feature (1)..(37): 3P Probe name 3P_Mseg_1 (detection)
  misc_feature (36)..(37): Diribonucleotides

SEQ ID NO: 212 - DNA, 37 bp, Influenza A virus; identical to SEQ ID NO: 54
  misc_feature (1)..(37): 5P Probe name 5P_Mseg_1 (detection)
  misc_feature (1)..(1): 5′ phosphorylated

SEQ ID NO: 213 - DNA, 37 bp, Influenza A virus
  misc_feature (1)..(37): 3P Probe name 3P_Mseg_2_A (detection)
  misc_feature (36)..(37): Diribonucleotides
ggagctgtcgttcactcctattgtatatgaggcccau

SEQ ID NO: 214 - DNA, 37 bp, Influenza A virus
  misc_feature (1)..(37): 5P Probe name 5P_Mseg_2_A (detection)
  misc_feature (1)..(1): 5′ phosphorylated
gcaactggcaagtgcaccagagatcggaagagcacac

SEQ ID NO: 215 - DNA, 37 bp, Influenza A virus
  misc_feature (1)..(37): 3P Probe name 3P_Mseg_2_G (detection)
  misc_feature (36)..(37): Diribonucleotides
ggagctgtcgttcactcctgttgtatatgaggcccau

SEQ ID NO: 216 - DNA, 37 bp, Influenza A virus; identical to SEQ ID NO: 214
  misc_feature (1)..(37): 5P Probe name 5P_Mseg_2_G (detection)
  misc_feature (1)..(1): 5′ phosphorylated

SEQ ID NO: 217 - DNA, 37 bp, Influenza A virus
  misc_feature (1)..(37): 3P Probe name 3P_Mseg_3_G (detection)
  misc_feature (36)..(37): Diribonucleotides
ggagctgtcgttcactctgagggcattttggataaag

SEQ ID NO: 218 - DNA, 37 bp, Influenza A virus
  misc_feature (1)..(37): 5P Probe name 5P_Mseg_3_G (detection)
  misc_feature (1)..(1): 5′ phosphorylated
cgtctacgctgcagtcctcgagatcggaagagcacac

SEQ ID NO: 219 - DNA, 37 bp, Influenza A virus
  misc_feature (1)..(37): 3P Probe name 3P_Mseg_3_T (detection)
  misc_feature (36)..(37): Diribonucleotides
ggagctgtcgttcactcttagggcattttggataaag

SEQ ID NO: 220 - DNA, 37 bp, Influenza A virus; identical to SEQ ID NO: 218
  misc_feature (1)..(37): 5P Probe name 5P_Mseg_3_T (detection)
  misc_feature (1)..(1): 5′ phosphorylated

SEQ ID NO: 221 - DNA, 37 bp, Influenza A virus; identical to SEQ ID NO: 49
  misc_feature (1)..(37): 3P Probe name 3P_OI_H1N1_P13L (detection)
  misc_feature (36)..(37): Diribonucleotides

SEQ ID NO: 222 - DNA, 37 bp, Influenza A virus; identical to SEQ ID NO: 50
  misc_feature (1)..(37): 5P Probe name 5P_OI_H1N1_P13L (detection)
  misc_feature (1)..(1): 5′ phosphorylated

SEQ ID NO: 223 - DNA, 37 bp, Influenza A virus; identical to SEQ ID NO: 51
  misc_feature (1)..(37): 3P Probe name 3P_OI_H1N1_P21L (detection)
  misc_feature (36)..(37): Diribonucleotides

SEQ ID NO: 224 - DNA, 37 bp, Influenza A virus; identical to SEQ ID NO: 52
  misc_feature (1)..(37): 5P Probe name 5P_OI_H1N1_P21L (detection)
  misc_feature (1)..(1): 5′ phosphorylated

SEQ ID NO: 225 - DNA, 37 bp, Influenza A virus; identical to SEQ ID NO: 55
  misc_feature (1)..(37): 3P Probe name 3P_OI_H1N1_P4L (detection)
  misc_feature (36)..(37): Diribonucleotides

SEQ ID NO: 226 - DNA, 37 bp, Influenza A virus; identical to SEQ ID NO: 56
  misc_feature (1)..(37): 5P Probe name 5P_OI_H1N1_P4L (detection)
  misc_feature (1)..(1): 5′ phosphorylated

SEQ ID NO: 227 - DNA, 37 bp, Homo sapiens
  misc_feature (1)..(37): 3P Probe name 3P_NM_002046_GAPDH_693 (detection)
  misc_feature (36)..(37): Diribonucleotides
ggagctgtcgttcactccatgagtccttccacgatac

SEQ ID NO: 228 - DNA, 37 bp, Homo sapiens
  misc_feature (1)..(37): 5P Probe name 5P_NM_002046_GAPDH_693 (detection)
  misc_feature (1)..(1): 5′ phosphorylated
caaagttgtcatggatgaccagatcggaagagcacac

SEQ ID NO: 229 - DNA, 20 bp, SARS-CoV-2
  misc_feature (1)..(20): Capture probe name cap_NC_045512.2_28655_28675
caacccatatgatgccgtct

SEQ ID NO: 230 - DNA, 20 bp, SARS-CoV-2
  misc_feature (1)..(20): Capture probe name cap_NC_045512.2_29057_29077
atgctttagtggcagtacgt

SEQ ID NO: 231 - DNA, 20 bp, SARS-CoV-2
  misc_feature (1)..(20): Capture probe name cap_NC_045512.2_28842_28862
tttcttgaactgttgcgact

SEQ ID NO: 232 - DNA, 20 bp, SARS-CoV-2
  misc_feature (1)..(20): Capture probe name cap_NC_045512.2_18017_18037
aaagttgccacattcctacg

SEQ ID NO: 233 - DNA, 20 bp, SARS-CoV-2
  misc_feature (1)..(20): Capture probe name cap_NC_045512.2_26997_27017
gcaggtccttgatgtcacag

SEQ ID NO: 234 - DNA, 20 bp, SARS-CoV-2
  misc_feature (1)..(20): Capture probe name cap_NC_045512.2_189_209
ggacgaaaccgtaagcagcc

SEQ ID NO: 235 - DNA, 20 bp, SARS-CoV-2
  misc_feature (1)..(20): Capture probe name cap_NC_045512.2_17695_17715
tttgtggcctgttaattgca

SEQ ID NO: 236 - DNA, 20 bp, SARS-CoV-2
  misc_feature (1)..(20): Capture probe name cap_NC_045512.2_461_481
tgatgaacacatagggctgt

SEQ ID NO: 237 - DNA, 20 bp, SARS-CoV-2
  misc_feature (1)..(20): Capture probe name cap_NC_045512.2_17801_17821
agtcccaaaatctttgaggc

SEQ ID NO: 238 - DNA, 20 bp, SARS-CoV-2
  misc_feature (1)..(20): Capture probe name cap_NC_045512.2_28085_28105
gggtgatttagaaccagcct

SEQ ID NO: 239 - DNA, 20 bp, SARS-CoV-2
  misc_feature (1)..(20): Capture probe name cap_NC_045512.2_14340_14360
gcatctgtcatccaaacagt

SEQ ID NO: 240 - DNA, 20 bp, SARS-CoV-2
  misc_feature (1)..(20): Capture probe name cap_NC_045512.2_23335_23355
aacactgacaccaccaaaag

SEQ ID NO: 241 - DNA, 20 bp, SARS-CoV-2
  misc_feature (1)..(20): Capture probe name cap_NC_045512.2_8713_8733
agatgctatgtcacgagtga

SEQ ID NO: 242 - DNA, 20 bp, SARS-CoV-2
  misc_feature (1)..(20): Capture probe name cap_NC_045512.2_11005_11025
tgtgagtaacaaccagtggt

SEQ ID NO: 243 - DNA, 20 bp, SARS-CoV-2
  misc_feature (1)..(20): Capture probe name cap_NC_045512.2_1318_1338
acaagtagtggcaccttctt

SEQ ID NO: 244 - DNA, 20 bp, SARS-CoV-2
  misc_feature (1)..(20): Capture probe name cap_NC_045512.2_26057_26077
acatgttcaacaccagtgtc

SEQ ID NO: 245 - DNA, 20 bp, SARS-CoV-2
  misc_feature (1)..(20): Capture probe name cap_NC_045512.2_14707_14727
agaaacccttagacacagca

SEQ ID NO: 246 - DNA, 20 bp, SARS-CoV-2
  misc_feature (1)..(20): Capture probe name cap_NC_045512.2_2938_2958
taaatcaatgcccagtggtg

SEQ ID NO: 247 - DNA, 20 bp, SARS-CoV-2
  misc_feature (1)..(20): Capture probe name cap_NC_045512.2_29577_29597
atcgtaaacggaaaagcgaa

SEQ ID NO: 248 - DNA, 20 bp, SARS-CoV-2
  misc_feature (1)..(20): Capture probe name cap_NC_045512.2_21653_21673
aaacaccacgtgtgaaagaa

(19) United States
(12) Patent Application Publication
(10) Pub. No.: US 2024/0401156 A1
(43) Pub. Date: Dec. 5, 2024

(54) HIGHLY MULTIPLEXED DETECTION OF NUCLEIC ACIDS

(71) Applicant: The Johns Hopkins University, Baltimore, MD (US)

(72) Inventors: Harry B. Larman, Baltimore, MD (US); Joel Credle, Gaithersburg, MD (US)

(21) Appl. No.: 17/767,389

(22) PCT Filed: Oct. 8, 2020
(86) PCT No.: PCT/US2020/054761

§ 371 Date: Apr. 7, 2022

Related U.S. Application Data
(60) Provisional application No. 62/912,238, filed on Oct. 8, 2019.

(51) Int. Cl. C12Q 1/70; C12Q 1/25; C12Q 1/6806; C12Q 1/6834; C12Q 1/6851

(52) U.S. Cl. CPC C12Q 1/701; C12Q 1/25; C12Q 1/6806; C12Q 1/6834; C12Q 1/6851

(57) ABSTRACT

The present invention relates to the field of ribonucleic acid (RNA). More specifically, the present invention provides compositions and methods for highly multiplexed detection of pathogen-associated RNA. In a specific embodiment, a method for forming a target ribonucleic acid (RNA) proxy in a sample comprises the steps of (a) contacting a sample with one or more multi-partite probes that hybridize to a target RNA, wherein the one or more multi-partite probes comprise (i) a target capture probe, (ii) a 3′acceptor probe and (iii) a 5′ phosphorylated donor probe; (b) incubating the sample of step (a) under conditions that allow hybridization of the one or more multi-partite probes to target RNA present in the sample; (c) immobilizing the target capture probes on a solid support; (d) washing away unbound multi-partite probes; and € ligating the acceptor probes and donor probes to form a target RNA proxy.

### CROSS-REFERENCE TO RELATED APPLICATIONS

This application claims the benefit of U.S. Provisional Application No. 62/912,238, filed Oct. 8, 2019, which is incorporated herein by reference in its entirety.

### GOVERNMENT SUPPORT CLAUSE

This invention was made with government support under grant nos. AI068613 and CA202875, awarded by the National Institutes of Health. The government has certain rights in the invention.

### FIELD OF THE INVENTION

The present invention relates to the field of ribonucleic acid (RNA) analysis. More specifically, the present invention provides compositions and methods for highly multiplexed detection of pathogen-associated RNA.

### INCORPORATION-BY-REFERENCE OF MATERIAL SUBMITTED ELECTRONICALLY

This application contains a sequence listing. It has been submitted electronically via EFS-Web as an ASCII text file entitled “P15983-02_ST25.txt.” The sequence listing is 79,933 bytes in size, and was created on Oct. 8, 2020. It is hereby incorporated by reference in its entirety.

### BACKGROUND OF THE INVENTION

Several RNA viruses have emerged in recent decades as threats to human health on a global scale (e.g., HIV, MERS, SARS, Ebola, Zika).^{1,2} In each case, the impact of these viruses would likely have been substantially mitigated by more effective surveillance technologies and contact tracing programs. In the early stages of the COVID-19 pandemic, widely available testing and contact tracing could have blunted the explosive growth of new cases. The public health crisis has been exacerbated by lack of critical supplies in some regions, including the RNA extraction kits required for reverse transcription polymerase chain reaction (RT-PCR) based molecular testing. At the time of this writing, the United States has reported nearly 2 million confirmed cases, over 100,000 COVID-19 related deaths, and over 40 million Americans have applied for unemployment. It is widely recognized that the development of a comprehensive testing infrastructure for large-scale diagnosis and surveillance, which is rapidly reconfigurable for emerging threats, is essential to ending the current pandemic and to preventing future crises due to emerging pathogens.

Current nucleic acid tests (NATs) for SARS-COV-2 have key limitations. Traditional RT-PCR is relatively inexpensive, but requires a separate labor and time-intensive RNA extraction step prior to nucleic acid amplification. Cartridge-based nucleic-acid tests offer rapid results and minimal sample preparation, but production of cartridges and low-throughput instruments limit scalability; further, most current testing platforms feature single pathogen targets and do not provide strain or clade-level information. Multiplexed PCR platforms and metagenomic next-generation sequencing (mNGS) technologies have demonstrated some advantages for diagnosing infections compared with more traditional approaches, but cost, low sensitivity, and complicated informatics have limited adoption for routine use.³ Platforms such as BioFire, Genmark ePlex and TaqMan array cards are able to identify up to 20-30 targets at a time, but their high per-sample costs (exceeding $100/test), as well as their inherently low sample throughput, severely limit their utility in the setting of the large-scale surveillance efforts.⁴⁻⁷ In the midst of the COVID-19 pandemic, innovative techniques involving targeted use of NGS have been reported, such as “SwabSeq” 8 and “LAMP-Seq”.⁹ While these methods are promising for detection of SARS-COV-2, they may not be well suited to syndromic panel (multiplex) testing or generalized surveillance, and may provide only limited clade-level information.

### SUMMARY OF THE INVENTION

The emergence of SARS-COV-2 has caused the current COVID-19 pandemic with catastrophic societal impact. Because many individuals shed virus for days before symptom onset, and many show mild or no symptoms, an emergent and unprecedented need exists for development and deployment of sensitive and high throughput molecular diagnostic tests. RNA-mediated oligonucleotide Annealing Selection and Ligation with next generation DNA sequencing (RASL-seq) is a highly multiplexed technology for targeted analysis of polyadenylated mRNA, which incorporates sample barcoding for massively parallel analyses. As described herein, the present inventors present a more generalized method, capture RASL-seq (“CRASL-seq”), which enables analysis of any targeted pathogen- (and/or host-) associated RNA molecules. In particular embodiments, cRASL-seq enables highly sensitive (down to ˜1-100 pfu/ml or cfu/ml) and highly multiplexed (up to ˜10,000 target sequences) detection of pathogens.

Importantly, cRASL-seq analysis, for example, of COVID-19 patient nasopharyngeal (NP) swab specimens, does not involve nucleic acid extraction or reverse transcription, steps that have caused testing bottlenecks associated with other assays. In certain embodiments, the simplified workflow additionally enables the direct and efficient genotyping of selected, informative SARS-COV-2 polymorphisms across the entire genome, which can be used for enhanced characterization of transmission chains at population scale and detection of viral clades with higher or lower virulence. Given its extremely low per-sample cost, simple and automatable protocol and analytics, probe panel modularity, and massive scalability, cRASL-seq testing is a powerful new surveillance technology with the potential to help mitigate the current pandemic and prevent similar public health crises.

In other embodiments, the present invention is applicable to detection of other pathogen-associated RNA. In fact, the present invention is applicable to detection of host RNAs as well. In particular embodiments, the present invention can be used to detect pathogen infection of patients, as well as detection of food contaminants, the presence of infectious organisms on fomites including, but not limited to, counter tops, tables, door handles, hand rails, as well as environmental samples, such as air filtration units, drinking water and sewage (for outbreak monitoring).

In particular embodiments, the techniques herein provide one or more multi-partite probes specific for target nucleic acid that may be contacted with a sample (e.g., patient sample, food product sample or sample from a surface). In certain embodiments, a multi-partite probe comprises a target nucleic acid capture probe, which binds to a portion of the target nucleic acid (e.g., a pathogen-associated RNA). A multi-partite probe may further comprise two or more ligation probes configured to anneal to a contiguous target sequence in the target nucleic acid, such that each of the two or more ligation probes binds the contiguous target sequence without leaving any unbound nucleotides in the contiguous target sequence between any of the two or more ligation probes that are adjacent. For example, in a specific embodiment, a multi-partite probe can comprise a target capture probe and two ligation probes. Once the two ligation probes are bound to the contiguous target sequence, the 3′ end of one of the ligation probes will be immediately adjacent to the 5′ end of the other ligation probe such that the two probes are bound in and end-to-end configuration. The target capture probe is bound to another part of the target nucleic acid. Once the multipartite probe is bound to the contiguous target sequence, the method further comprises capturing the target nucleic acid capture probe, which is bound to the target nucleic acid along with the two ligation probes. Then, a ligase (e.g., T4 RNA Ligase 2 (Rnl2)) may be used to ligate the adjacent, bound probes to create a target nucleic acid proxy (e.g., a pathogen-associated RNA proxy).

In one aspect, the present invention provides compositions and methods useful for forming a target ribonucleic acid (RNA) proxy in a sample. In a specific embodiment, a method comprises the steps of (a) contacting a sample with one or more multi-partite probes that hybridize to a target RNA, wherein the one or more multi-partite probes comprise (i) a target capture probe, (ii) a 3′ acceptor probe and (iii) a 5′ phosphorylated donor probe; (b) incubating the sample of step (a) under conditions that allow hybridization of the one or more multi-partite probes to target RNA present in the sample; (c) immobilizing the target capture probes on a solid support; (d) washing away unbound multi-partite probes; and (e) ligating the acceptor probes and donor probes to form a target RNA proxy.

In another specific embodiment, the method further comprises amplifying the target RNA proxy, wherein the 3′ acceptor probe and the 5′ phosphorylated donor probe comprise amplification primer binding sites. The method can further comprise detecting the target RNA by one or more of sequencing, quantitative PCR, microarray hybridization, toe-hold amplification, and loop-mediated isothermal amplification. In particular embodiments, the amount of target RNA is quantified.

In certain embodiments, the 3′ acceptor probe comprises at least one 3′ terminal ribonucleotide. In a more specific embodiment, the 3′ acceptor probe comprises a 3′ terminal diribonucleotide.

In particular embodiments, the target RNA is a pathogen-associated RNA. In a specific embodiment, the sample is obtained from a patient suspected of being infected by a pathogen. In alternative embodiment, the sample is obtained from a food product. In another embodiment, the sample is obtained from a fomite. In certain embodiments, the amount of pathogen is quantified.

In another specific embodiment, the 3′ acceptor probe and/or the 5′ phosphorylated donor probe comprise alternative single nucleotide polymorphisms (SNPs) of the target RNA to enable genotype identification. In particular embodiments, the one or more multi-partite probes are designed for multiplex detection of one or more target RNAs. In certain embodiments, the sample is obtained from a human and the one or more multi-partite probes are designed to detect human RNA.

In a specific embodiment, a method for forming a target pathogen-associated RNA proxy in a sample obtained from a patient suspected of being infected with a pathogen comprises the steps of (a) contacting a sample obtained from the patient with one or more multi-partite probes that hybridize to a target pathogen-associated RNA, wherein the one or more multi-partite probes comprise (i) a target capture probe, (ii) a 3′ acceptor probe and (iii) a 5′ phosphorylated donor probe; (b) incubating the sample of step (a) under conditions that allow hybridization of the one or more multi-partite probes to target pathogen-associated RNA present in the sample; (c) immobilizing the target capture probes on a solid support; (d) washing away unbound multi-partite probes; and (e) ligating the acceptor probes and donor probes to form a target pathogen-associated RNA proxy.

In another specific embodiments, the method further comprises amplifying the target pathogen-associated RNA proxy, wherein the 3″ acceptor probe and the 5′ phosphorylated donor probe comprise amplification primer binding sites. In yet another specific embodiment, the method further comprise detecting the target pathogen-associated RNA by one or more of sequencing, quantitative PCR, microarray hybridization, toe-hold amplification, and loop-mediated isothermal amplification.

In certain embodiments, the amount of pathogen is quantified. In another embodiment, the 3′ acceptor probe and/or the 5′ phosphorylated donor probe comprise alternative SNPs of the target pathogen-associated RNA to enable genotype identification. In yet another embodiment, the one or more multi-partite probes are designed for multiplex detection of one or more pathogens. In other embodiments, at least one of the one or more multi-partite probes are designed to detect patient RNA.

In a specific embodiment, a method for detecting pathogen contamination of food products or fomites comprises the steps of (a) contacting a sample obtained from a food product or fomite with one or more multi-partite probes that hybridize to a target pathogen-associated RNA, wherein the one or more multi-partite probes comprise (i) a target capture probe, (ii) a 3′ acceptor probe and (iii) a 5′ phosphorylated donor probe; (b) incubating the sample of step (a) under conditions that allow hybridization of the one or more multi-partite probes to target pathogen-associated RNA present in the sample; (c) immobilizing the target capture probes on a solid support; (d) washing away unbound multi-partite probes; (e) ligating the acceptor probes and donor probes to form a target pathogen-associated RNA proxy; and (f) detecting the target pathogen-associated RNA proxy to identify the pathogen.

In certain embodiment, detecting step (f) comprises sequencing, quantitative PCR, microarray hybridization, toe-hold amplification, and loop-mediated isothermal amplification. In particular embodiments, the amount of pathogen is quantified. In other embodiments, the 3′ acceptor probe and/or the 5′ phosphorylated donor probe comprise alternative SNPs of the target pathogen-associated RNA to enable genotype identification. In certain embodiments, the one or more multi-partite probes are designed for multiplex detection of one or more pathogens.

In another specific embodiment, a method for detecting a severe acute respiratory syndrome coronavirus 2 (SARS-COV-2) infection in a patient comprises the steps of (a) contacting a patient sample with one or more multi-partite probes that hybridize to a target SARS-COV-2-associated RNA, wherein the one or more multi-partite probes comprise (i) a target capture probe, (ii) a 3″ acceptor probe and (iii) a 5′ phosphorylated donor probe; (b) incubating the sample of step (a) under conditions that allow hybridization of the one or more multi-partite probes to target SARS-COV-2-associated RNA present in the sample; (c) immobilizing the target capture probes on a solid support; (d) washing away unbound multi-partite probes; (e) ligating the acceptor probes and donor probes to form a target SARS-COV-2-associated RNA proxy; and (f) detecting the target SARS-COV-2-associated RNA proxy. thereby detecting a SARS-COV-2 infection in the patient.

In particular embodiments, detecting step (f) comprises sequencing, quantitative PCR, microarray hybridization, toe-hold amplification, and loop-mediated isothermal amplification. In one embodiments, the amount of SARS-COV-2 is quantified.

In some embodiments, the 3′ acceptor probe and/or the 5′ phosphorylated donor probe comprise alternative SNPs of the target SARS-COV-2-associated RNA to enable genotype identification. In other embodiments, the one or more multi-partite probes are designed for multiplex detection of one or more pathogens. In specific embodiments, at least one of the one or more multi-partite probes are designed to detect patient RNA.

In another specific embodiment, the one or more multi-partite probes comprise one or more of SEQ ID NOS:171-180. In other embodiments, (i) the 3′ acceptor probe comprise one or more of SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, SEQ ID NO:163, SEQ ID NO:165, SEQ ID NO:167, and SEQ ID NO:169; (ii) the 5′ phosphorylated donor probe comprises one or more of SEQ ID NO:108, SEQ ID NO:110, SEQ ID NO:112. SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170. In certain embodiments, the target capture probe comprises one or more of SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, and SEQ ID NO:200.

In particular embodiments, the labeled target capture probe comprises biotin, diogexin, acrydite, haloalkane, or click chemistry. In other embodiments, the solid support comprises a capture element selected from the group consisting of avidin, streptavidin, neutravidin, anti-digoxin antibodies, click chemistry, halo protein, or a combination thereof. In further embodiments, the solid support comprises magnetic material, polystyrene, agarose, silica, lateral flow strip, microfluidic chambers, or a combination thereof.

In certain embodiments, the ligating step is performed using an enzyme selected from the group consisting of T4 RNA Ligase 2 (Rnl2), a Chlorella virus DNA ligase (PBCV-1 DNA Ligase), a T4 DNA Ligase, derivatives thereof, and combinations thereof.

In particular embodiments, the pathogens comprise one or more of Coronavirus HKU1, Coronavirus NL63, Coronavirus 229E, Coronavirus OC43, Middle East Respiratory Syndrome Coronavirus (MERS-COV, Severe Acute Respiratory Syndrome Coronavirus 2 (SARS-COV-2), Influenza A/H1, Influenza A/H3, Influenza A/H1-2009, Parainfluenza Virus 1, Parainfluenza Virus 2, Parainfluenza Virus 3, Parainfluenza Virus 4, Bordetella pertussis, Chlamydophila pneumoniae, Enterococcus faecalis, Enterococcus faecium, Staphylococcus aureus, Staphylococcus epidermidis, Staphylococcus lugdunensis, Streptococcus agalactiae, Streptococcus pyogenes, Streptococcus pneumoniae, Candida albicans, Candida auris, Candida glabrata, Candida krusei, Candida parapsilosis, Candida tropicalis, Campylobacter (jejuni, coli & upsaliensis), Clostridium difficile (Toxin A/B), Plesiomonas shigelloides, Salmonella, Yersinia enterocolitica, Vibrio (parahaemolyticus, vulnificus, & cholerae), Vibrio cholerae. E. coli O157, Enteroaggregative E. coli (EAEC), Enteropathogenic E. coli (EPEC), Enterotoxigenic E. coli (ETEC) lt/st. Shiga-like toxin-producing E. coli (STEC) stx1/stx2 E. coli O157, Shigella/Enteroinvasive E. coli (EIEC), Adenovirus F 40/41, Astrovirus, Norovirus GI/GII, Rotavirus A, Sapovirus (I,II, IV, and V), Cryptosporidium, Cyclospora cayetanensis, Entamoeba histolytica, Giardia lamblia, Escherichia coli K1, Haemophilus influenzae, Listeria monocytogenes, Neisseria meningitidis, Streptococcus agalactiae, Streptococcus pneumoniae, Cytomegalovirus (CMV), Enterovirus, Herpes simplex virus 1 (HSV-1), Herpes simplex virus 2 (HSV-2), Human herpes virus 6 (HHV-6), Human parechovirus, Varicella zoster virus (VZV), Cryptococcus neoformans/gattii, Acinetobacter calcoaceticus-baumannii complex, Enterobacter cloacae, Escherichia coli, Klebsiella aerogenes, Klebsiella oxytoca, Klebsiella pneumoniae group, Moraxella catarrhalis, Proteus spp., Pseudomonas aeruginosa, Serratia marcescens, Staphylococcus aureus, Streptococcus agalactiae, Streptococcus pyogenes, Legionella pneumophila, Mycoplasma pneumoniae, Chlamydia pneumoniae, Influenza A, Influenza B, Adenovirus, Coronavirus, Parainfluenza virus, Respiratory Syncytial virus, Human Rhinovirus/Enterovirus, Human Metapneumovirus, and Middle East Respiratory Syndrome Coronavirus (MERS-COV).

In another aspect, the present invention provides kits for performing the methods described herein. In one embodiment, a kit comprises a SARS-COV-2 probe set comprising one or more multi-partite probes comprising SEQ ID NOS:171-180. In another embodiment, the kit further comprises one or more multi-partite SARS-COV-2 SNP probes comprising one or more of SEQ ID NOS:107-170. In yet another embodiment, the kit further comprise one or more SARS-COV-2 capture probes comprising one or more of SEQ ID NOS:181-200.

In another embodiment, a kit comprises a Candida albicans probe set comprising one or more multi-partite probes comprising SEQ ID NOS:3-4 and SEQ ID NOS: 81-82. In yet another embodiment, a kit comprises a Cryptococcus neoformans probe set comprising one or more multi-partite probes comprising SEQ ID NOS:15-26 and SEQ ID NOS:83-86.

In a specific embodiment, a kit comprises a Haemophilus influenza probe set comprising one or more multi-partite probes comprising SEQ ID NOS:27-32 and SEQ ID NOS: 87-88. In another specific embodiment, a kit comprises a human cytomegalovirus probe set comprising one or more multi-partite probes comprising SEQ ID NOS:33-48 and SEQ ID NOS:89-92.

In a further embodiment, a kit comprises an influenza type A probe set comprising one or more multi-partite probes comprising SEQ ID NOS:49-56 and SEQ ID NOS:93-94. In another embodiment, a kit comprises an mycobacterium smegmatis probe set comprising one or more multi-partite probes comprising SEQ ID NOS:57-62 and SEQ ID NOS:95-96.

In a specific embodiment, a kit comprises a Pseudomonas aeruginosa probe set comprising one or more multi-partite probes comprising SEQ ID NOS:63-68 and SEQ ID NOS:97. In a further embodiment, a kit comprises Staphylococcus aureus probe set comprising one or more multi-partite probes comprising SEQ ID NOS:69-74.

In yet another embodiment, a kit comprises Zika virus probe set comprising one or more multi-partite probes comprising SEQ ID NOS:75-80 and SEQ ID NOS:99-100.

### BRIEF DESCRIPTION OF THE FIGURES

FIG. 1A-1F. The cRASL-seq method. FIG. 1A: A ligation probe set is composed of a chimeric DNA-RNA 3′ acceptor probe and a phosphorylated 5′ donor probe. 20 nt target recognition sequences bring these probes adjacent to one another on a target RNA, enabling their enzymatic ligation. A biotinylated capture probe is included to separate the target sequence from irrelevant materials and excess ligation probes. FIG. 1B: cRASL/RASL-seq complementary assays, which can be performed in a single reaction. FIG. 1C: Sample (e.g., NP swab specimen) is added to lysis buffer containing cRASL probes. After lysis and annealing, targets are captured for subsequent ligation and sample-barcoding amplification, followed by amplicon pooling and NGS. FIG. 1D: Amount of ligation product formed on transcribed GAPDH RNA as a function of input amount; analysis by qPCR of ligation product. FIG. 1E: cRASL-seq test on a set of 9 blinded NP swabs (unextracted) from 6patients with influenza A and 3 negative controls. FIG. 1F: Assay performed as in FIG. 1E, with influenza capture probe doped into a background of irrelevant capture probe at the ratio shown. For FIG. 1D-1F, Molecular Equivalents are calculated by normalizing to a PCR spike-in sequence of defined copy number input.

FIG. 2A-2H. Universal cRASL-seq assay for pathogen-associated RNA analysis. Each reference organism was serially diluted into PBS and added directly to the lysis buffer and probe pool. NLC, No Ligase Control; NTC, No Template Control. The extraction free protocol of FIG. 1C was performed with all 116 probe sets in a single pool. Molecular Equivalents are calculated by normalizing read counts to a PCR spike-in sequence of known copy number. Detection at a signal >10× the NTC was used to calculate the assay's limit of detection for each organism.

FIG. 3A-3E. Multiplexed SNP genotyping of SARS-COV-2 gRNA directly from unextracted NP swabs. FIG. 3A: A probe pair is designed with SNP position in the middle of the 5′ phospho donor probe. A base-calling algorithm is applied to the reads from each alternative probe. FIG. 3B: 1 of 20 positions had a base call for the reference Washington isolate, which matched perfectly against the known genotype. FIG. 3C: The 3 samples from the set of 40 PCR+ samples analyzed, which had 5 or more base calls (♦). Red indicates mutant and blue indicates wildtype versus the reference Wuhan seafood market isolate. FIG. 3D: Network graph depicts each observed genotype (each individual node), two of which are linked if they do not have conflicting SNPs at any position. The blue nodes indicate a maximal vertex set of independent genotypes detected among the 3 samples that passed QC. FIG. 3E: Comparison between reads from a SNP typing cRASL-seq probe set in the N gene, versus the Ct value from the RT-qPCR (n=37, 3 samples missing Ct values).

FIG. 4A-4I. Quantification of host panel RASL-seq data. Eight NP swab specimens were subjected to RASL-seq in duplicate. Normalized read counts are plotted for each probe set.

FIG. 5. RASL-seq measurement of host immune gene expression directly from unextracted COVID-19 patient swab specimens. Results from quantification of the immunoglobulin family of genes (IgA, IgD, IgE, IgG, IgM) and a housekeeping target, β-actin, are shown. Sums of normalized read counts from all probe sets targeting the Ig or β-actin transcript are plotted (y-axis) for samples with decreasing SARS-COV-2 viral load (increasing RT-qPCR Ct values left to right, labeled below the axis).

FIG. 6A-6C. Optimization of the cRASL-seq assay. 1,000 pfu of N1N1 influenza virus collected via nasopharyngeal swab was used in standard cRASL reactions together with probe sets targeting the influenza M-segment. All reactions were analyzed by qPCR probes specific for the ligated product. FIG. 6A: Effects on RNA templated and non-templated ligation efficiency with varying Rnl2 amounts (0, 7.5, 15, and 30 units) and Rnl2 ligation times (5, 10, and 20 minutes). FIG. 6B: Effects of varying post-ligation wash steps and sample-reagent reaction storage times. FIG. 6C: Effects of varying hybridization times (20, 30, and 60 minutes). NTC, no template control. Wash, 1x-SSC. BE, buffer exchange into Rnl2 reaction buffer. Premade, complete cRASL reaction stored for 24 hours at room temperature prior to start of assay. All reactions were performed in duplicate, averages are plotted.

FIG. 7A-7B. Network graph analysis of replica SNP-genotyping data. Nodes represent sample genotypes and are linked if they do not have conflicting SNPs at any position (as in FIG. 3D). Technical replicas are distinguished by color. Samples lacking sufficient called SNPs were not included (FIG. 7A: 5 SNPs to pass QC, FIG. 7B: 3 SNPs to pass QC).

FIG. 8A-8B. Alignment of detected SARS-COV-2 genotypes against the GISAID database. FIG. 8A: Comparison of 20 SNP genotypes (columns) detected in FIG. 3C against genotypes present in the GISAID database (rows). Rows were hierarchically clustered; column order is maintained from FIG. 3C. A match (box shaded black) means there were no conflicting SNPs between the sample and GISAID genotype. Independent genotypes (1-9) and reference isolate (♦) are indicated below, as in FIG. 3C. FIG. 8B: Heatmap linking GISAID genotypes (rows, same order as in FIG. 8A) with geographic location (columns), requiring exact matching to at least one isolate from the geographic region.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Analysis of pathogen-associated RNA, versus DNA, can be valuable for several reasons. A large fraction of clinically important viruses, such as coronaviruses, have RNA genomes, and many have no DNA stage of their lifecycle. All of the viral NIAID Emerging Infectious Diseases Category A and B pathogens are RNA viruses.¹⁰ Further, viral mRNA can also be detected from DNA viruses that cause disease, and may provide a diagnostic advantage over DNA testing by distinguishing between active and latent infections.^{11,12} For cellular pathogens, abundant RNA sequences, such as ribosomal RNA sequences, provide biological amplification compared to analysis of the organism's genomic DNA, thereby enhancing detection sensitivity.^{13,14} In addition, RNA typically degrades rapidly outside of cells, permitting differentiation between living organisms and environmental/reagent contaminants. Compared with DNA, RNA tends to be shorter and usually exists in single stranded form, making it more amenable to techniques involving probe hybridization. Finally, simultaneous analysis of viral and host mRNA expression has been shown to provide additional, clinically useful diagnostic and prognostic information about disease states.¹⁵-17 Importantly, the RNA analysis method presented herein avoids nucleic acid extraction, which is an advantage since limited supplies of the reagents needed for this step of analysis has contributed to the disruption of large-scale SARS-COV-2 testing efforts in the United States.¹⁸

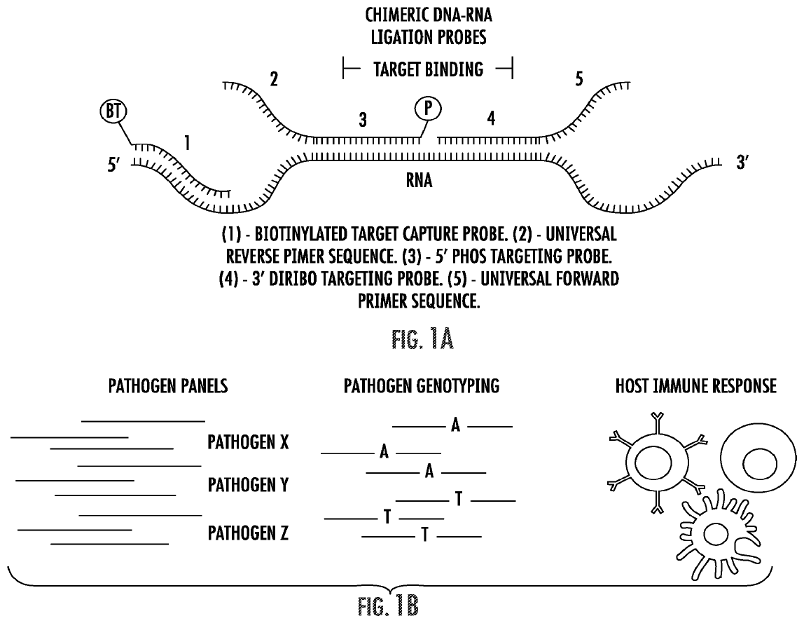

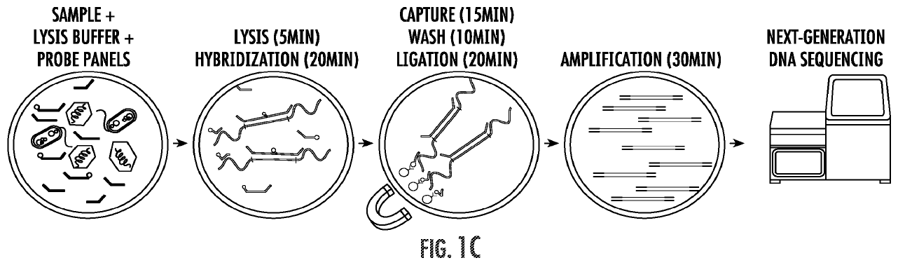

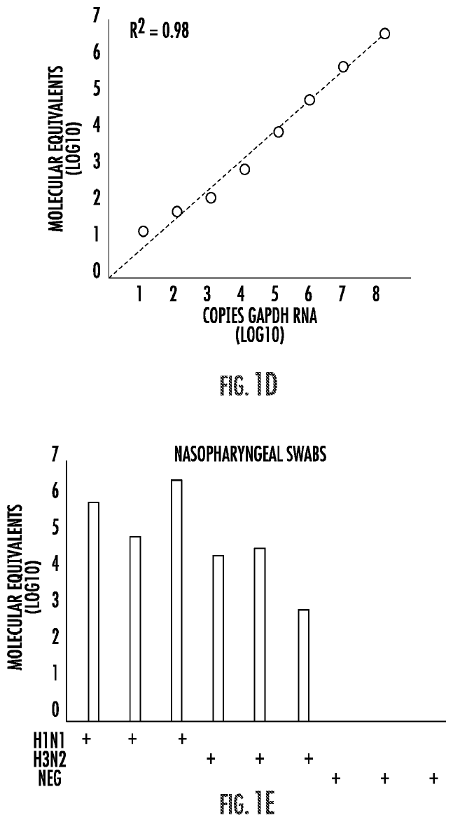

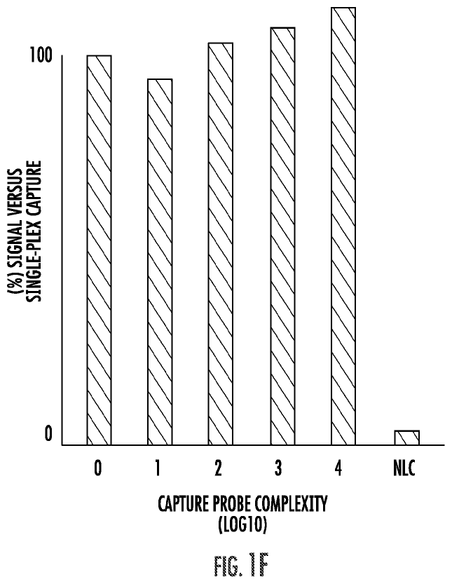

Several years ago, the present inventors and others described a modified RNA-mediated oligonucleotide Annealing, Selection, and Ligation with next-generation sequencing (RASL-seq) assay chemistry with enhanced sensitivity. This efficient reaction utilizes, in certain embodiments, the T4 RNA Ligase 2 (Rnl2), which despite being an RNA ligase, efficiently catalyzes ligation of a DNA donor probe and a chimeric acceptor probe composed of two bases of ribonucleotides at the ligation junction (FIG. 1A).¹⁹⁻²³ In addition to the high sensitivity required for pathogen detection, RASL-seq also enables very high levels of probe set multiplexing, potentially providing the means for simultaneous analysis of pathogens, their ancestral lineages, and host immune response (FIG. 1B). By incorporating DNA barcodes into the primers used to amplify the ligation products, a high level of sample multiplexing is achievable, which enables very high sample throughput and extremely low per-sample cost.

In any probe ligation assay, it is important that excess probe be removed or destroyed in order to reduce the amount of non-specific background probe ligation.^{24,25,26} In contrast to previously published methods, the present inventors have incorporated the oligonucleotide-mediated capture of target nucleic acid molecules, in an assay the present inventors refer to as “capture RASL-seq” or “cRASL-seq” (FIG. 1C). By separating targeted from untargeted RNA molecules, and thus, hybridized from un-hybridized ligation probes, cRASL-seq permits extremely high assay specificity, which is especially important in the setting of, for example, diagnosing infectious diseases-particularly relevant in the early phases of an emerging pathogen threat when community prevalence is low. This method of target capture is distinct from RASL-seq analysis, which relies on immobilized oligo-dT for non-specific capture of polyadenylated mRNA.

The present inventors have previously demonstrated that libraries of ligation products are amplified with uniform efficiency,²⁵ so that PCR spike-ins enable precise quantification of the copies of each ligation product formed prior to amplification. Quantification of target molecules has proven useful in clinical settings, in determining the burden of organism(s) within a clinical specimen. Furthermore, as described herein, the present inventors demonstrate how cRASL-seq probes can be used for simultaneous SARS-COV-2 detection and SNP genotyping, which has utility for tracking chains of viral transmission. Recognizing the urgent need for large-scale testing at minimal cost, the present inventors have optimized and characterized the performance of a streamlined, extraction-free protocol for direct analysis of nasopharyngeal (NP) swab specimens obtained from COVID-19 patients.

### I. Definitions

“Detect” refers to identifying the presence, absence, or amount of the nucleic acid (e.g., RNA) to be detected.

By “detectable label” is meant a composition that when linked to a molecule of interest renders the latter detectable, via, for example, spectroscopic, photochemical, biochemical, immunochemical, or chemical means. For example, useful labels may include radioactive isotopes, magnetic beads, metallic beads, colloidal particles, fluorescent dyes, electron-dense reagents, enzymes (for example, as commonly used in an ELISA), biotin, digoxigenin, or haptens.

By “fragment” is meant a portion of a nucleic acid molecule or polypeptide. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “marker” is meant any protein or polynucleotide having an alteration in expression level or activity that is associated with a disease or disorder. The term “biomarker” is used interchangeably with the term “marker.”

By “multi-partite” is meant having several or many parts or divisions.

By “multi-partite probe set” is meant a probe set having multiple parts or divisions. As an example, a multi-partite probe set of the present invention may include a 3′ acceptor probe, a 5′ donor probe, and a biotinylated target capture probe.

By “pathogen” is meant anything that can produce a disease including a bacterium, virus, fungi or other microorganism, as examples.

By “infection” is meant the invasion of an organism's body by disease-causing agents, their multiplication and the reaction of the host to these organisms and the toxins they produce. The infection may be caused by any microbes/microorganisms, including for example, bacteria, fungi, and viruses. Microorganisms can include all bacterial, archaean, and the protozoan species. This group also contains some species of fungi, algae, and certain animals. In some embodiments, viruses may be also classified as microorganisms.

By “reduces” is meant a negative alteration of at least 10%, 25%, 50%, 75%, or 100%.

By “reference” is meant a standard or control conditions such as a sample (human cells) or a subject that is a free, or substantially free, of an agent such as a pathogen.

By “reference sequence” is meant a defined sequence used as a basis for sequence comparison. A reference sequence may be a subset of or the entirety of a specified sequence; for example, a segment of a full-length cDNA, RNA, or gene sequence, or the complete cDNA, RNA, or gene sequence. For polypeptides, the length of the reference polypeptide sequence will generally be at least about 16 amino acids, preferably at least about 20 amino acids, more preferably at least about 25 amino acids, and even more preferably about 35amino acids, about 50 amino acids, or about 100 amino acids. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 nucleotides or any integer thereabout or there between.

By “sensitivity” is meant a percentage of subjects correctly identified as having a particular disease or pathogen.

By “specificity” is meant a percentage of subjects correctly identified as NOT having a particular disease or pathogen, i.e., normal or healthy subjects.

By “specifically binds” is meant a multi-partite probe set that recognizes and binds a nucleotide sequence of the invention, but which does not substantially recognize and bind other molecules in a sample, for example, a biological sample, which naturally includes nucleotide sequences unrelated to the invention. In some embodiments, a genotyping probe specifically binds a target nucleic acid having a particular single nucleotide polymorphism (SNP), but does not specifically bind a nucleic acid having an alternative SNP.

By “subject” is meant any individual or patient to which the method described herein is applied. Generally, the subject is human, although as will be appreciated by those in the art, the subject may be an animal (e.g. pet, agricultural animal, wild animal, etc.), disease vector (e.g. mosquitoes, sandflies, triatomine bugs, blackflies, ticks, tsetse flies, mites, snails, lice, etc.), or an environmental sample (e.g. sewage, food products, etc.). Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Nucleic acid molecules useful in the methods of the invention need not be 100% identical with an endogenous nucleic acid sequence, but will typically exhibit substantial identity. Polynucleotides having “substantial identity” to an endogenous sequence are typically capable of hybridizing with a target molecule. By “hybridize” is meant pair to form a double-stranded molecule between complementary polynucleotide sequences, or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and sometimes above 50° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Sequencing” or any grammatical equivalent as used herein may refer to a method used to sequence the amplified target nucleic acid proxy. The sequencing technique may include, for example, Next Generation Sequencing (NGS), Deep Sequencing, mass spectrometry based sequence or length analysis, or DNA fragment sequence or length analysis by gel electrophoresis or capillary electrophoresis. Compatible sequencing techniques may be used including single-molecule real-time sequencing (Pacific Biosciences), Ion semiconductor (Ion Torrent sequencing), pyrosequencing (454), sequencing by synthesis (Illumina), sequencing by ligation (SOLID sequencing), chain termination

(Sanger sequencing), Nanopore DNA sequencing (Oxford Nanosciences Technologies), Helicos single molecule sequencing (Helicos Inc.), sequencing with mass spectrometry, DNA nanoball sequencing, sequencing by hybridization, and tunneling currents DNA sequencing.

By “NGS” is meant Next Generation Sequencing. NGS platforms perform massively parallel sequencing, during which millions of fragments of DNA from a single sample are sequenced in unison. Massively parallel sequencing technology facilitates high-throughput sequencing, which allows an entire genome to be sequenced in less than one day. The creation of NGS platforms has made sequencing accessible to more labs, rapidly increasing the amount of research and clinical diagnostics being performed with nucleic acid sequencing.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705,BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

“Primer set” means a set of oligonucleotides that may be used, for example, in a polymerase chain reaction (PCR). A primer set comprises at least 2, 4, 6, 8, 10, 12, 14, 16, 18, 20, 30, 40, 50, 60, 80, 100, 200, 250, 300, 400, 500, 600, or more primers.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the term “sub-probe” may refer to any of the two or more probes that bind the contiguous target sequence without leaving any unbound intervening nucleotides. In some embodiments, the multi-partite probe described herein may include at least two “sub-probes.” In another embodiment, each of the at least two sub-probes of the plurality of multi-partite probes may be about 10-50 nucleotides in length. Once the probes are ligated, the ligated multi-partite probe (alternatively, the “ligated sub-probe”) may be released from the RNA. In some embodiments, the sub-probe may contain appended primer binding site (e.g., adapters) to facilitate subsequent amplification of the target nucleic acid proxy. In other embodiments, at least one of the two or more sub-probes may be referred to as “acceptor sub-probes” that have a 3′-termination of at least two RNA bases.

As used herein, “appended primer binding” sites may refer to binding sites within the multi-partite probe or sub-probes described herein that facilitate amplification of the target nucleic acid proxy. “Appended primer binding sites” may also be referred to as “adapters.”

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing.” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### II. CRASL-seq

The present disclosure provides compositions and methods for analyzing the presence and expression level of nucleic acids in a sample. In particular embodiments, the presence and expression level of RNA is analyzed. In certain embodiments, the sample is from a patient suspected of having a pathogen infection. In other embodiments, the sample is from a food product that is suspected of being contaminated, for example, by a pathogen. Food items include, but are not limited to, produce (e.g., vegetables, fruit, etc.) and meat. In further embodiments, the sample to be analyzed is a surface of an inanimate object (e.g., counter tops, tables, door handles, hand rails, etc.). The present invention can also be used in an environmental context, for example, samples can be obtained from air filtration units, drinking water, sewage—for outbreak monitoring, etc.).

In one aspect, the disclosure provides a method comprising contacting a sample with one or more multi-partite probes, wherein a multi-partite probe comprises (i) a target capture probe and (ii) at least two sub-probes or ligation probes (e.g., an acceptor probe and a donor probe, as described herein), annealing at least one of the contacted one or more multi-partite probes to at least one target RNA within the sample, capturing the target capture probe (bound to a target nucleic acid along with at least two sub-probes or ligation probes), and ligating the at least two sub-probes associated with the at least one annealed multi-partite probe to create a target nucleic acid proxy that can be detected. The method may further comprise releasing the target nucleic acid proxy from the target nucleic acid. The method may also comprise amplifying the target nucleic acid proxy, which method may comprise a heating step that releases the target nucleic acid proxy from the target nucleic acid. In particular embodiments, each probe comprises an oligonucleotide, e.g., DNA, RNA, or a mixture of both DNA and RNA. In particular embodiments, the target nucleic acid is RNA (e.g., a pathogen-associated RNA and/or host-RNA).

In one embodiment, the sub-probes (ligation probes) comprise appended primer binding sites which facilitate the post-ligation amplification of the target nucleic acid proxy.

In certain embodiments, each sub-probe comprises an oligonucleotide. In a specific embodiment, the sub-probes have a 3′-termination of at least two RNA bases (which may also be referred to as “acceptor sub-probes” or “acceptor ligation probes”). In a more specific embodiment, the sub-probe/ligation probe is a DNA oligonucleotide that has a 3′-termination of at least two RNA bases. In other embodiments, the sub-probe is a DNA oligonucleotide that has a 5′-phosphorylation (which may also be referred to as “donor sub-probes” or “donor ligation probes”).

In certain embodiments, the at least two sub-probes may be ligated with an enzyme, a chemical reaction, or a photoreaction. The enzyme may be a ligase. In particular embodiments, the enzyme may be one of the following ligases: a T4 RNA Ligase 2 (Rnl2), T4 DNA Ligase, a Chlorella virus DNA Ligase (PBCV-1 DNA Ligase), a Rnl2 derivative, PBCV-1 derivative, or any combination thereof.

In an embodiment, the sample may be a cell. In some embodiments, the sample is manipulated prior to contacting with the labeled target capture probe and the one or more multi-partite probes. For example, a cell may be lysed prior to such step.

In some embodiments, the target nucleic acid proxy (e.g., a pathogen-associated RNA proxy) may be released from the target nucleic acid by an endonuclease or recovered by denaturing the target nucleic acid containing the at least two ligated sub-probes. The endonuclease may be RNaseH, RNaseA, RNase If, or RNaseHIII. Alternatively, an amplification step comprises a heat denaturing step that releases the proxy from the target.

In specific embodiments, the target nucleic acid proxy may be amplified using PCR. In an embodiment, the PCR includes about 20-50 cycles, e.g., about 20, about 25, about 30, about 35, about 40), about 45, or about 50 cycles.

In particular embodiments, several techniques can be used on the amplified target nucleic acid proxy to detect/identify the target nucleic acid. Such techniques include, but are not limited to, Next Generation Sequencing (NGS), Deep Sequencing, mass spectrometry based sequence or length analysis, DNA fragment sequence or length analysis by gel electrophoresis or capillary electrophoresis, hybridization on immobilized detection probes qPCR, microarray hybridization, toe-hold amplification, LAMP, etc.

### III. Pathogens

The methods described herein are useful for diagnosing a pathogen infection, e.g., viral infection, bacterial infection, or fungal infection. For example, a sample is obtained from a subject suspected of or at risk of developing a viral infection, a bacterial infection, or a fungal infection. The target nucleic acid is selected from the group consisting of a viral nucleic acid, a bacterial nucleic acid, and a fungal nucleic acid. In some embodiments, the method further comprises releasing the target nucleic acid proxy; amplifying the target nucleic acid proxy; and sequencing the target nucleic acid proxy, thereby identifying a viral nucleic acid, a bacterial nucleic acid or a fungal nucleic acid and diagnosing a viral infection, a bacterial infection, or a fungal infection, respectively, in the subject. The method may also include quantifying the amount of pathogen or otherwise, determining the severity of the infection. In further embodiments, after diagnosis, the subject is treated with an anti-fungal agent, an anti-bacterial agent, or an anti-viral agent.

Fungal infections may include, but are not limited to, infections derived from the following organisms: Acremonium sp., Aspergillus clavatus, Aspergillus flavus, Aspergillus fumigatus, Aspergillus glaucus, Aspergillus nidulans, Aspergillus niger, Aspergillus ochraceus, Aspergillus terreus, Aspergillus unguis, Aspergillus ustus, Beauveria sp., Bipolaris sp., Blastoschizomyces sp., Blastomyces dermatitidis, Candida albicans, Candida glabrata, Candida guilliermondii, Candida kefyr, Candida krusei, Candida lusitaniae, Candida parapsilosis, Candida tropicalis, Chrysosporium sp., Cladosporium sp., Coccidioides immitis, Cryptococcus neoformans var gattii serotype B, Cryptococcus neoformans serotype A, Cryptococcus laurentii, Cryptococcus terreus, Curvularia sp., Fusarium sp., Filobasidium capsuligenum, Filobasidiella (Cryptococcus) neoformans var bacillispora serotype C, Filobasidiella (Cryptococcus) neoformans var neoformans serotype D, Filobasidium uniguttulatum, Geotrichum sp., Histoplasma capsulatum, Malbranchea sp., Mucor sp., Paecilomyces sp., Paracoccidioides brasiliensis, Penicillium species, Pneumocystis carinii, Pseudallescheria boydii, Rhizopus sp., Sporothrix schenkii, Scopulariopsis brevicaulis sp., Scopulariopsis brumpti, Saccharomyces cerevisiae, and Trichosporon beigelii.

Bacterial infections include, but are not limited to, infections derived from the following organisms: Bacillus anthracis, Bordetella pertussis, Borrelia burgdorferi, Brucella abortus, Brucella canis, Brucella melitensis, Brucella suis Brucellosis, Campylobacter jejuni, Chlamydia pneumoniae respiratory infection, Chlamydia psittaci, Chlamydia trachomatis, Lymphogranuloma venereum, Clostridium botulinum, Clostridium difficile, Clostridium perfringens, Clostridium tetani, Corynebacterium diphtheria, Enterococcus faecalis, Enterococcus faecium, Escherichia coli, Francisella tularensis, Haemophilus influenza, Helicobacter pylori, Legionella pneumophila, Leptospira interrogans, Listeria monocytogenes, Mycobacterium leprae, Mycobacterium tuberculosis, Mycoplasma pneumonia, Neisseria gonorrhoeae, Neisseria meningitides, Pseudomonas aeruginosa, Rickettsia, Salmonella typhi, Salmonella typhimurium, Shigella sonnei, Staphylococcus aureusa, Staphylococcus epidermidis, Staphylococcus saprophyticus, Streptococcus agalactiae, Streptococcus pneumonia, Streptococcus pyogenes, Treponema pallidum, Vibrio cholera, and Yersinia pestis.

Viral infections include, but are not limited to, infections derived from the following organisms: Adenoviruses, Avian influenza, Influenza virus type A, Influenza virus type B, Measles, Parainfluenza virus, Respiratory syncytial virus RSV), Rhinoviruses, SARS-COV (Severe Acute Respiratory Syndrome), SARS-COV-2, Coxsackie viruses, Enteroviruses, Poliovirus, Rotavirus, Hepatitis B virus, Hepatitis C virus, Bovine viral diarrhea virus (surrogate), Herpes simplex 1, Herpes simplex 2, Human cytomegalovirus, Varicella zoster virus, Human immunodeficiency virus 1 (HIV-1), Human immunodeficiency virus 2 (HIV-2), Simian immunodeficiency virus (SIV), Simian human immunodeficiency virus (SHIV), Dengue virus, Hantavirus, Hemorrhagic fever viruses, Lymphocyti, choromeningitis virus, Smallpox virus surrogates (Cowpox, Monkeypox, Rabbitpox), Vaccinia virus, Venezuelan equine encephalomyelitis virus (VEE), West Nile virus, Yellow fever virus, and Zika virus.

### IV. Multi-Partite Probes

Multi-partite probes present a way of bringing together and localizing two or more nucleic acid sequences. A multi-partite probe may comprise a target capture probe, which is used to capture the target nucleic acid after annealing with the target capture probe and at least two ligation probes. Such probes include a target nucleic acid binding sequence capable of hybridizing with the target genetic sequence and an additional nucleic acid binding probe sequence adjacent to the target nucleic acid binding sequence. For example, multi-partite probes may each include two or more nucleic acid probes configured to anneal to a contiguous target sequence in a target nucleic acid (e.g., a RNA), such that each of the two or more probes bind the contiguous target sequence without leaving any unbound nucleotides in the contiguous target sequence between any of the two or more probes that are adjacent (e.g., a sub-probe).

Each sub-probe within the multi-partite probe may range from 10-200 bases in length. For example, 10-200 is understood to include any number, combination of numbers or sub-range of numbers, as well “nested sub ranges” that extend from either end point of the range. For example, a nested sub-range of an exemplary range of 10 to 200 may comprise 10 to 20,to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190 and 10 to 200.

In specific embodiments, the at least two sub-probes of the plurality of multi-partite 10 probes may be about 10-200 nucleotides in length, e.g., about 10, about 15, about 20, about 25, about 30, about 35, about 40, about 50, about 75, about 100, about 125, about 150, about 175, or about 200 nucleotides in length. In another embodiment, each of the at least two sub-probes of the plurality of multi-partite probes may be about 15-30 nucleotides in length, e.g., about 15, about 16, about 17, about 18, about 19, about 20, about 21, about 22, about 23, about 24, about 25, about 26, about 27, about 28, about 29, or about 30 nucleotides in length.

In a further embodiment, each of the plurality of multi-partite probes comprises two sub-probes or ligation probes. In a related embodiment, each of the plurality of multi-partite probes comprises three or more sub-probes.

### V. Multiplexing

In particular embodiments, the cRASL-seq technique described herein may be amenable to a highly multiplexed RNA analysis method. Multiplex gene expression analysis provides direct and quantitative measurement of multiple nucleic acid sequences simultaneously using a detection system. Multiplex assays utilize a strategy where more than one target is amplified and quantified from a single sample aliquot. In multiplex PCR, a sample aliquot is queried with multiple probes that contain fluorescent dyes in a single PCR reaction. This increases the amount of information that can be extracted from the sample. With the cRASL-seq technique described herein, hundreds to thousands of probes can be analyzed at the same time. For example, it is contemplated within the scope of the invention that a typical sample may be analyzed with 10-10,000 multi-partite probes simultaneously.

### VI. Ligation

Rnl2 (dsRNA Ligase) is an ATP-dependent dsRNA ligase that efficiency seals 3′-OH/5′PO₄ nicks in duplex RNAs. This process occurs via adenylation of the ligase, AMP transfer to the 5′PO₄ on the donor strand, and the attack by the acceptor strand 3′-OH on the 5′-adenylylated donor strand, resulting in the formation of the covalent phosphodiester linkage. Rnl2 tolerates complete substitution of its duplex RNA substrate with deoxyribonucleotides, provided that the 3′-terminus of the acceptor strands terminates in a diribonucleotide. The technique employs fully deoxyribonucleotide donor probes with 5′-PO₄ termini, which undergo highly efficient, template-dependent ligation to hybrid deoxyribonucleotide-3′-diribonucleotide acceptor probes. In summary, Rnl2 can ligate RNA probes or RNA-DNA hybrid probes in which one probe has the 3′ two bases as RNA when annealed on either RNA or DNA templates. Rnl2 cannot efficiently ligate fully DNA probes annealed on DNA templates or fully DNA probes on RNA templates.

The ligase may be introduced to join adjacently annealed acceptor and donor probe sets. Enzymatic ligation covalently joins the probes, which then serves as a template for PCR-based signal amplification. Under typical conditions, all components of the ligation reaction are in excess over the target RNA, ensuring the direct proportionality between template molecules and ligation events. Subsequently, the ligation products may be amplified and barcoded during a PCR amplification step.

It is contemplated within the scope of the disclosure that other types of ligases may be used according to the techniques herein including, but not limited to, T4 DNA ligase and PBCV-1 DNA Ligase (Chlorella virus DNA Ligase).

### RNaseH (Ribonuclease H)

### VII. Ribonuclease H (RNase H)

In certain embodiments, once probes are ligated, the ligated probe (e.g., the sub-probes) (the target nucleic acid proxy) may be released from the RNA template so that it may be recovered and used as an amplification source. In other embodiments, the amplification step comprises a heat denaturation step that accomplishes the release step above. According to the techniques herein, RNaseH may be used to release the ligated multi-partite probe (e.g., the sub-probes). RNAseH belongs to a family of non-sequence-specific endonucleases that catalyze the cleavage of RNA via a hydrolytic mechanism. Members of the RNase H family may be found in nearly all organisms, from bacteria to archaea to eukaryotes. Because RNaseH specifically degrades only the RNA in RNA:DNA hybrids, it is commonly used in molecular biology to destroy the RNA template after first-strand cDNA synthesis by reverse transcription, as well as in procedures such as nuclease protection assays. RNase H can also be used to degrade specific RNA strands when the cDNA oligonucleotide is hybridized, such as the removal of the polyadenine tail from mRNA hybridized to oligo (dT), or the destruction of a chosen non-coding RNA inside or outside the living cell. RNaseH specifically hydrolyzes the phosphodiester bonds of RNA, which is hybridized to DNA. This enzyme does not digest single or double-stranded DNA. To terminate the reaction, a chelator, such as EDTA, is often added to sequester the metal ions in the reaction mixture, or the enzyme can be heat destroyed.

Following ligation of adjacently annealed probes, the ligation product (target nucleic acid proxy) may be retrieved by incubation of the sample with RNAseH, which destroys the RNA component of the RNA/DNA hybrid helices and releases the ligated sub-probes into solution. The product may then be amplified and analyzed to detect the target nucleic acid (e.g., by sequencing). Alternative methods to recover the ligated probe sets include, but are not limited to other RNase enzymes (such as RNase A, RNase I_{f}, RNase HII, for example), thermal treatment to melt the hybrid helices or mechanical tissue extraction (e.g., by laser capture microdissection or scraping) prior to PCR amplification.

### VIII. Probes and Probe Design

Multi-partite probes can be designed with a probe set design pipeline similar to Primer-BLAST and implemented using Primer 3, BLASTN, Melting, pandas and the Python standard library. Custom Primer3 settings can be implemented to design up to 20 separate 36nucleotide probes antisense to the target transcript. Primer3-designed probes can be extended, for example, 4 base pairs in the 5′ direction of the probe (towards the poly(A)tail). Each 40 nucleotide sequence can be then split in half and common adaptors (ADI for acceptor probes and RCAD2 for donor probes) can be appended. Primer3 can be then called to calculate the properties of each probe oligo plus adaptor. Empirically derived thresholds for the Primer3 calculations can be used to filter the candidate probe. Remaining probes with an off-target Tₘ within 10° C. of the predicted on-target melting temperature can be removed. A non-parametric ranking scheme using distance to poly(A)tail and Primer3 penalty (based on the original 36 nucleotide Primer3-design probe) can be then employed to select the two predicted best probe sets annealing at least 10 nucleotide distance from one another for each target transcript. The acceptor oligo 3′-terminal and 3′-penultimate bases is changed to their RNA counterparts.

Critical parameters of probe design include the probe length and the melting temperature of the annealing sequences. For a given target sequence, increasing the probe length increases the strength of the specific binding interaction, but may also increase inappropriate ligations by non-specific binding to off-target sequences and/or decrease the probe's effective concentration in the reaction.

A library of donor and acceptor probes was created to explore the impact of transcript annealing sequence length, ranging from 12 to 22 nucleotides. Junction positions were kept constant in order to eliminate potentially confounding variables such as nucleotide sequence bias. For both donor and acceptor probes, the on-target ligation yield depended on the length of the probe. Because probe cost increases with length, and because diminishing improvements were observed in relative quantification after about 20 nucleotides for most probes, a probe design algorithm was developed to identify adjacent 20 nucleotide sequences in target transcripts. However, it is contemplated within the scope of the disclosure that the length of the probes within a multi-partite probe may range from 10-200 nucleotides in length.

A probe decoy strategy was developed to reduce the sampling of transcripts expressed at very high levels to optimize the efficiency of sequence analysis. Each probe set is flanked by a common primer binding sequence, so that ligation products can be separately amplified by primers containing a short DNA barcode specific for each well. Decoy probes lack the primer binding sequences, so that they form un-amplifiable ligation products at desired levels. PCR products from multiple samples can thus be pooled together for sequencing and individual reads subsequently deconvoluted by their corresponding barcodes. The finite number of DNA sequencing reads obtained results in oversampling of highly abundant transcripts. The probe decoy strategy overcomes this sampling bias.

The compositions of the present invention can be used to detect and/or genotype pathogens. In particular embodiments, the present invention is directed to the detection and/or genotyping of coronaviruses. Coronaviruses include, but are not limited to, 229E, SARS-COV, SARS-COV-2, NL63, HKU1, MERS-COV and OC43. In one embodiment, detection probes for human coronavirus 229E comprise one or more of SEQ ID NOS:101-106. In another embodiment, genotyping probes for SARS-COV-2 (which could be used for detection, as well) comprise one or more of SEQ ID NOS:107-170. In a further embodiment, detection probes for SARS-COV-2 comprise one or more of SEQ ID NOS:171-180. In certain embodiments, SARS-COV-2 target capture probes comprise SEQ ID NOS:229-248.

In another embodiment, probes for detection of human coronavirus NL63 comprise SEQ ID NOS:181-186. In yet another embodiment, probes for SARS-COV comprise SEQ ID NOS:187-192. Probes for human coronavirus HKUI may comprise SEQ ID NOS:193-198. In a specific embodiment, probes for MERS-COV comprise SEQ ID NOS:199-204. In yet another embodiment, probes for human coronavirus OC43 comprise SEQ ID NOS:205-210.

In particular embodiments, probes for Influenza-A comprise SEQ ID NOS:211-226. Probes for detection of candida albicans comprise one or more of SEQ ID NOS:3-14. Capture probes for candida albicans may comprise one of SEQ ID NOS:81-82.

In other embodiments, probes for detection of Cryptococcus neformans comprise one or more of SEQ ID NOS:15-26. Capture probes for detection of Cryptococcus neoformans may comprise one or more of SEQ ID NOS:83-86.

In a specific embodiment, probes for detection of Haemophilus influenza comprise one or more of SEQ ID NOS:27-32. Capture probes for detection of haemophilus influenza comprise one of SEQ ID NOS:87-88.

In another specific embodiment, probes for detection of human cytomegalovirus comprise one or more of SEQ ID NOS:33-48. Capture probes for detection of human cytomegalovirus may comprise one or more of SEQ ID NOS:89-92.

In yet another embodiment, probes for detection of Influenza Type A comprise one or more of SEQ ID NOS:49-56. Capture probes for detection of Influenza Type A may comprise one of SEQ ID NOS:93-94.

In a further embodiment, probes for detection of Mycobacterium smegmatis comprise one or more of SEQ ID NOS:57-62. Capture probes for detection of Mycobacterium smegmatis may comprise one of SEQ ID NOS:95-96.

In particular embodiments, probes for detection of Pseudomonas aeruginosa comprise one or more of SEQ ID NOS:63-68. Capture probes for detection of Pseudomonas aeruginosa may comprise SEQ ID NO:97.

In other embodiments, probes for detection of Staphylococcus aureus comprise one or more of SEQ ID NOS:69-74.

In certain embodiments, probes for detection of Zika virus comprise one or more of SEQ ID NOS:75-80. Capture probes for Zika virus detection may comprise one or more of SEQ ID NOS:98-100.

The capture probes described above can be labeled as described herein including, but not limited to, biotin, diogexin, acrydite, haloalkane, or click chemistry. In particular embodiments, the label is biotin.

Examples of probes can be found in Supplemental Tables 2-3 of Credle et al., “Highly multiplexed oligonucleotide probe-ligation testing enables efficient extraction-free SARS-CoV-2 detection and viral genotyping”, which can be on the bioRxiv website (posted Jun. 3, 2020). Such probes and accompanying information are incorporated by reference herein. The capture probes described in the sequence listing can be labeled as described herein.

The 3′ probes described in the sequence listing and in Credle et al (2020) indicate that, in certain embodiments, diribonucleotides are present. However, the invention is not so limited. Indeed, a standard ligation of the ends of the 3′ and 5′ probe can be conducted, using no modified nucleotides. Other chemistries are known in the art and can be used.

### IX. Host RNA Detection

In particular embodiments. the compositions and methods of the present invention can be used to detect host or patient RNAs (reference genes or expression (e.g., immune response)). Examples of such RNAs include, but are not limited to, RNA transcribed from the following genes: ARG1, CD274, CD4, CD8A, CSFIR, CTLA4, EBI3, FOXP3, GAPDH, GATA3, GUSB, GZMB, HAVCR2, HIF1A, HPRT1, ICOSLG, IDO1, IDO2, IFNG, IKZF2, IKZF4, IL10, IL10RA, IL12A, IL13, IL17A, IL18, IL1B, IL21, IL22, IL23A, IL23R, IL6, IRF4, LAG3, MMP9, NOS2, PDCD1, PRF1, PTPRC, RORC, TBX21, TGFB1, TNF, VEGFA, TMX2, CIITA, ENTPD1, AFP, BCL2L11, BID, CD101, TTF1, BCL2L1, CD244, CD276, CD86, COX20, PSMB10, SLAMF6, TAGAP, TGFB2, TIA1, VTCN1, ADORA2A, CD34, CSF1, EPCAM, GPC3, HNF1A, KRT7, NT5E, TGFBR2, TMEM173, TNFRSF14, TNFRSF9, BAX, CD14, CD33, CD44, CD80, CMKLR1, EOMES, HLA-E, IL2RG, TGFB3, TOX, VSIR, AIF1, ATRAID, CCR5, CD226, CD38, CD3D, CD3E, CD68, CD70, CD79A, IL17F, IL25, PDCD1LG2, PECAM1, PTGER4, TNFRSF18, TNFRSF4, TNFSF9, BAK1, BTLA, CCR2, CD19, CD2, CD40LG, CXCR4, CXCR6, GZMA, GZMK, ITGAL, LGALS9, NKG7, SNAI1, XBP1, BIK, CCL5, CD163, CD27, CD40, CXCL10, CXCL11, CXCL13, CXCL9, ERG, IFNB1, IL5, NLRP3, PDGFRA, SLAMF7, SLC4A3, TIGIT, TNFSF14, TNFSF4, BAD, CD28, IFNA1, STAT1, NCAM1, ITGAM, SDC1, B3GAT1, NCR1, PROM1, MS4A1, CCR4, ITGAX, MKI67, CCL4, CCR10, CCR6, CCR7, CD24, CD47, CD5, CD74, CD83, CD8B, CDK5R1, CLEC4C, CR2, CSF2, CXCR5. EMR1, FAS, FCGR3A, FUT4, HLA-DR. IL12B, IL2RA, IL3RA, IL7R, ITGA2, KI, KLRK1, MME, MRC1, NCR3, NGFR, NRP1, PTPN13, SELL, SIRPA, TCRA, TCRBC1, TCRGC2, TFRC, THBD, TNFRSF8, UCHL1, CD69, CXCR3, IL2, ITGB3, POUSF1, LAMP1, RPL19, PTGS2, B2M, ABCF1, ACTB, ALAS1, Clorf43, CHMP2A, CLTC, EMC7, G6PD, GPI, LDHA, PGK1, POLR1B, POLR2A, PSMB2, PSMB4, RAB7A, REEP5, RPLP0, SDHA, SNRPD3, TBP, TUBB, VCP, VPS29, and TMX2. Examples of probes for such genes can be found in Supplemental Table 1 of Credle et al., “Highly multiplexed oligonucleotide probe-ligation testing enables efficient extraction-free SARS-COV-2 detection and viral genotyping”, which can be on the bioRxiv website (posted Jun. 3. 2020). Such probes are incorporated by reference herein. In particular embodiments, a host RNA for detection (e.g., for quality control) comprises GAPDH. In a specific embodiments, probes for GAPDH detection comprise one of SEQ ID NOS:227-228.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Probe design and synthesis. For each target sequence from the reference organisms (target sequences described in Results, and Table 1), the present inventors identified 40-mer sites for ligation probes using CATCH.³⁰ To avoid overlapping probes, the present inventors set a stride of 40, allowing no mismatches and bypassing the cover extension in the design.py program (-pl 40-1 40-ps 40-m 0-e 0). The present inventors excluded probes that aligned against any target sequences from the other organisms, with an e-value smaller than 10-3, using MMseqs2.³¹ A similar design pipeline was employed for 20-mer capture probes with a final filter step to remove any overlapping ligation and capture probes. Finally, ligation and capture probes were filtered for binding properties using previously reported Primer3 conditions.¹⁹ The design of the immunoglobulin gene expression probe panel was reported previously.¹⁹ Ligation probes and capture probes (3′-diribonucleotide terminated acceptor probes, 5′ phosphorylated probes, and biotinylated capture probes) were synthesized by Integrated DNA Technologies (Coraville, IA 52241, USA). Probes were diluted in water to 100 μM, mixed in equimolar amounts to create multiplexed panels, and then aliquoted and stored at −80° C. (Supplemental Tables 1-3 of Credle et al., “Highly multiplexed oligonucleotide probe-ligation testing enables efficient extraction-free SARS-COV-2 detection and viral genotyping”, which can be on the bioRxiv website (posted Jun. 3, 2020); sequence listing). The reference genome that we have used for probe set coordinate harmonization is NC_045512.2.

TABLE 1

| Organism/Target | Sample Type | Source |
|---|---|---|
| Human GAPDH RNA | In vitro transcribed | Thermo Fisher Ultimate ORF Collection |
| | RNA | Catalog# IOH3380 |
| Candida albicans | Clincal isolate | ATCC Strain ID 18804 |
| Cryptococcus neoformans | Clincal isolate | ATCC Strain ID 32045 |
| Pseudomonas aeruginosa | Clincal isolate | ATCC Strain ID 10145 |
| Haemophilus influenzae | Clincal isolate | ATCC Strain ID 10211 |
| Mycobacterium smegmatis | Clincal isolate | ATCC Strain ID 1448 |
| Staphylococcus aureus | Clincal isolate | ATCC Strain ID 25923 |
| Influenza virus Type A | Nasopharyngeal | Johns Hopkins Influenza Research and |
| | swab | Surveillance Program |
| Zika virus | Clincal isolate | Emergent Neuroviruses in the Americas |
| | | Study-NEAS |
| Human cytomegalovirus | Clincal isolate | ATCC Strain ID VR-38 |
| SARS-CoV-2 samples | NP swab | Johns Hopkins University School of Medicie, |
| 1-50 | | Department of Pathology & Medical Microbioloy |
| SARS-CoV-2 RNA | Purified RNA from | Johns Hopkins University School of Medicie, |
| | NP swab | Department of Pathology & Medical Microbioloy |

Spike-ins and reference organisms. The synthetic PCR spike-in sequence used for determining molecular equivalents is a 74 nt oligo with a pseudo 40 nt ligated sequence flanked by the external 17 nt PCR1 primer binding sites: 5′-GGAGCTGTCGTTCACTCTGTCTCGGAGCTTACAGTrArUTGACACTCAATCGGTCG CGTAGATCGGAAGAGCACAC-3′) (SEQ ID NO:1). The 40 nt irrelevant internal sequence is a scrambled version of a ligated GAPDH probe set. Reference organisms were purchased from American Type Culture Collection (ATCC, Manassas, VA) (Table 1). Organisms were reconstituted according to protocols provided by ATCC, aliquoted into single-use samples and stored at −80° C. until used. The Zika virus isolate was from a patient in Cali, Colombia, and was grown in Vero-E6 cells. The infectious titer of the virus (7.1×10⁶ pfu/mL) was determined in the culture supernatant by a plaque assay. The full-length GAPDH ORF (RefSeq: NM_002046.6) was subcloned into a custom vector, linearized and transcribed in vitro using the Hi-Scribe T7 High Yield RNA Synthesis Kit (NEB, Ipswich, MA). GAPDH IVT-RNA was purified by precipitation with lithium chloride followed by column purification using the RNeasy Mini Kit (Qiagen, Hilden, DE). Purified GAPDH IVTRNA was quantified by nanodrop, aliquoted into single-use samples and stored at −80° C. until used for the dynamic range experiments.

Nasopharyngeal swab specimens. NP swab specimens were collected from patients after informed written consent was obtained, under a protocol approved by the local governing human research protection committee. The Johns Hopkins Influenza Research and Surveillance (JH-CEIRS) program's human subjects protocol was approved by the Johns Hopkins School of Medicine Institutional Review Board (IRB): IRB90001667 and NIH Division of Microbiology and Infectious Diseases: Protocol 15-0103. Unextracted NP swab specimens (n=9) were de-identified, blinded and provided for further analysis. Secondary use of all unextracted COVID-19 patient NP swab specimens (n=40) was exempted by the Johns Hopkins University School of Medicine Institutional Review Board protocol (IRB00086059, and IRB00221396, Table 1). An RT-qPCR diagnostic test (RealStar® from Altona Diagnostics. Hamburg. Germany)³² was performed on all COVID-19 patient NP swab specimens used in this study. NP swab specimens were stored either at −20° C. or −80° C.

CRASL and RASL assays. Samples (39 μL) were added to 61 μL of a hybridization reaction mix containing 1XSSC. 5 pM of each ligation and capture probe. 40 U of Protector RNase Inhibitor (Roche Diagnostics. Mannheim. Germany) and 50 μL of 2X DNA/RNA Shield (Zymo Research, Irvine CA) in a total reaction volume of 100 μL. Reactions were heated for 5 min at 95° C. followed by 20 min annealing at 45° C. In other embodiments, the reactions were heated for 5 min at 95° C. and allowed to cool on bench top to 25° C. and incubated for an additional 4 min. 5 μL of a 50/50 slurry of streptavidin coated magnetic beads (Dynabeads MyOne C1. Thermo Fisher Scientific. Waltham MA) in 1x-PBS was added to each cRASL reaction and incubated with gentle shaking for 15 min at room temperature. To each RASL reaction. 5 μL of Oligo-(dT) 25 beads (Dynabeads. Thermo Fisher Scientific. Waltham MA) were added and incubated with gentle shaking for 15 min at 25° C. Beads were collected on a magnet for 2 minutes and washed twice with 1X-SSC buffer. followed by a final wash with 1X Rnl2 reaction buffer (50) mM Tris-HCl, 10 mM MgCl₂. 5 mM DTT, 1 mM ATP, pH 7.6). 10 μL of the ligation reaction containing 30 U of Rnl2 (Enzymatics. Beverly MA) in 1X Rnl2 buffer was incubated with the beads in suspension for 20 min at 37° C. Following ligation, beads were collected for 2 min on a magnet and then resuspended in 25 μL PCR master mix containing PCRI primers and Herculase-II (Agilent. Santa Clara CA). In other embodiments, the beads were incubated a final time on the magnet for 2 min to collect the beads, discarding the supernatant followed by addition to individual reactions 20 μL PCT master mix reactions using universal primers. PCR cycling was as follows: an initial denaturing step at 95° C. for 2 min, followed by 30 cycles of: 95° C. for 20 s, 53.5° C. for 30 s. 72° C. for 30 s, with a final extension of 72° C. for 3 min. Two microliters of the pre-amplification product were used as input to a 20 μL dual-indexing PCR reaction for 10 cycles with primers containing 8-mer i5 and 8-mer i7 barcodes and the P5/P7 Illumina adapters. PCR cycling was as follows: an initial denaturing step at 95° C. for 2 min, followed by 10 cycles of: 95° C. for 20 s, 58° C. for 30 s, 72° C. for 30 s, with a final extension of 72° C. for 3 min.

Library preparation and sequencing. Barcoded PCR products were analyzed individually or as a pool on a 3% agarose gel to confirm amplicon size and purity. Barcoded PCR products were pooled and purified using NucleoSpin PCR Clean-up columns (Mackery Nagel, Duren DE). Pooled libraries were sequenced on an Illumina NextSeq 500 instrument (Illumina, La Jolla CA), using a single-end 50-cycle protocol with a custom read 1 sequencing primer and custom i5/i7 sequencing primers as previously described.¹⁷

Quantification of ligation products. Relative quantification of ligated products (molecular equivalents) were calculated using a synthetic PCR spike-in (described above), which was added to PCR1 reactions at 3,000 or 5,000 molecules/reaction. For samples measured by qPCR, molecular equivalents were calculated using the following equation: 2^{-(Ctspike-Ctligation)Nspike} where Nₛₚᵢₖₑ is the molecules of spike-in added to PCR1 reactions. For samples analyzed by sequencing, a pseudocount was added to each probe set's read count and the molecular equivalents were calculated by taking the ratio of ligation product read count to spike-in read count, multiplied by the molecules of spike-in added to the PCR1 reaction. For comparison of read counts with Ct values, the present inventors performed baseline subtraction on spike-in normalized probe values by subtracting the maximum normalized value of each probe from the four negative samples (two no ligase controls and two seasonal coronavirus samples). The corrected values of the best performing genotyping probe set, which targets the N gene at genome position 28,688, was plotted against clinically-determined SARS-COV-2 RT-qPCR Ct values. The present inventors fit an exponential regression to the data, and graphed the resulting data points and regression on a semilog plot. For analysis of host gene expression, psuedocounted and GAPDH-normalized values were obtained for each probe set. For the analysis of Ig gene expression, the normalized values of all probe sets corresponding to each Ig (3 probes each for IGHA, IGHD, IGHE, and IGHM, and 8 probes for IGHG1-4) were summed to obtain the final values plotted in FIG. 5.

Analysis of sequencing data. Sequencing reads were trimmed to the first 40 bases, demultiplexed and aligned against a reference database of the intended ligation products using exact matching. Genotyping data was analyzed as follows. Genotyping probe pairs were evaluated using an exact binomial test with a null model of equal abundance between the wild type probe and the mutant probe. A SNP was called if the binomial p-value was <0.001, the total reads mapped to the probe pair was >100, and the fold change between the probes was >1.5. A locus with read counts that failed any of these criteria was not called, and thus considered a “wildcard”. Each set of 20 SNP calls and wildcards is referred to as the SARS-COV-2 genotype associated with a given sample. The present inventors utilized a network graph-based approach to visualize the relationships of genotypes detected in the Baltimore COVID-19 cohort. Genotypes were represented as nodes and were connected if there was no disagreement between the pair of genotypes (wildcards could match anything). The present inventors utilized the R package igraph to determine which set of samples would result in the largest number of unique genotypes (the “maximal independent vertex set”).

### Results

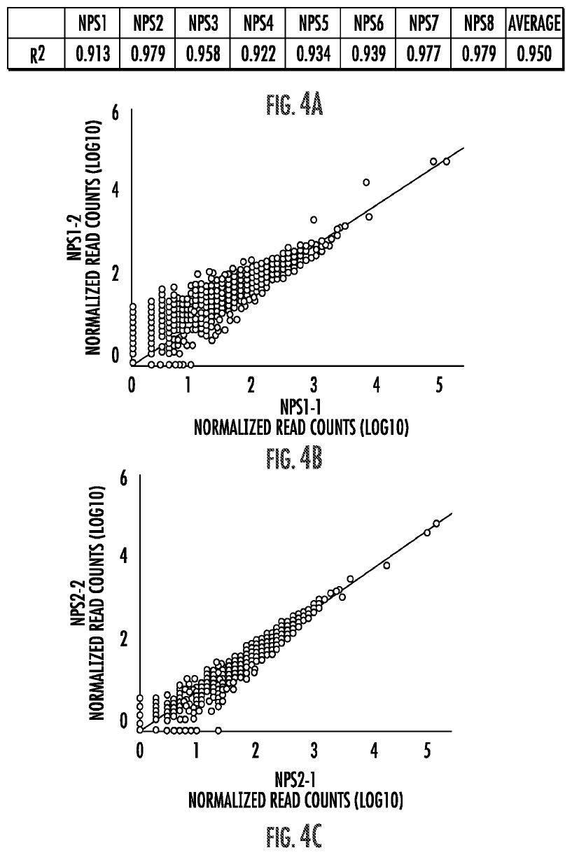

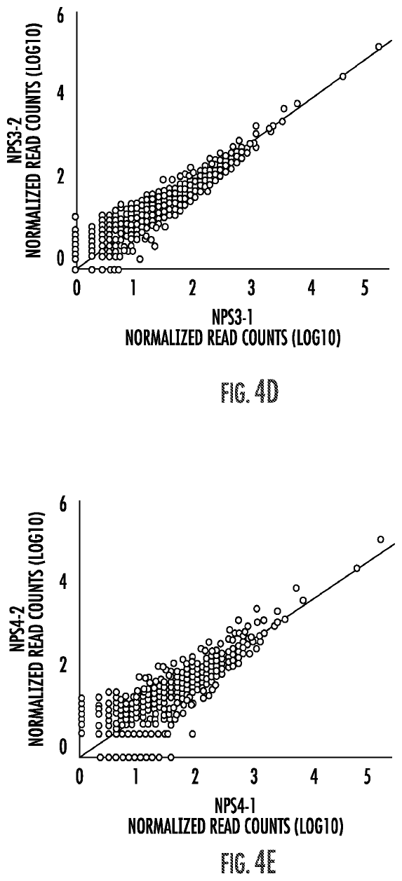

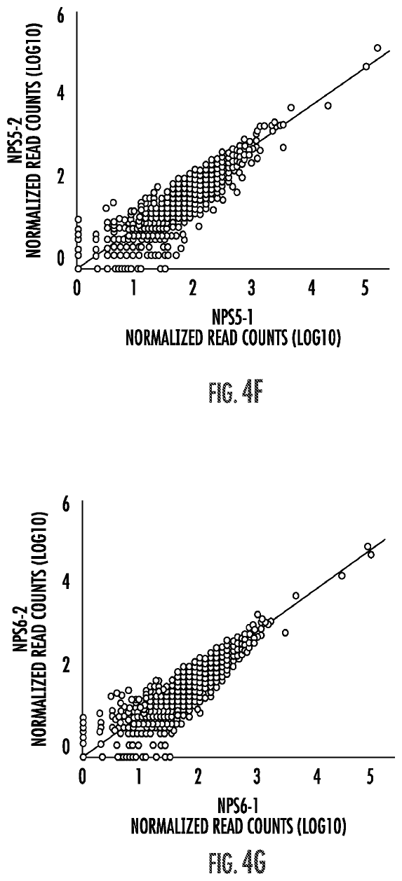

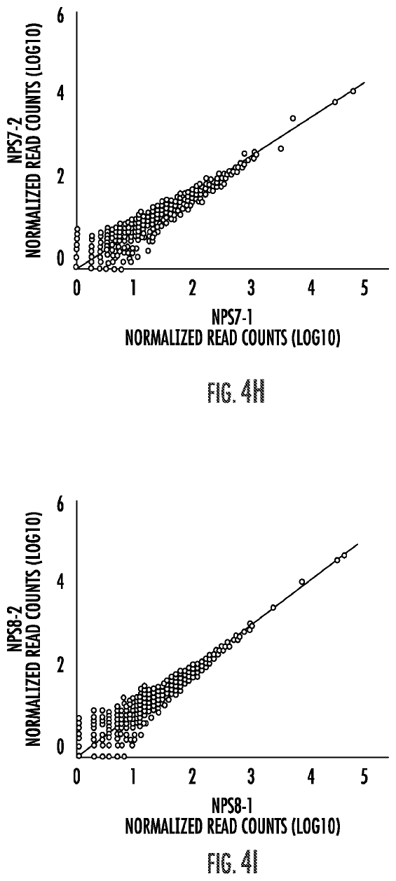

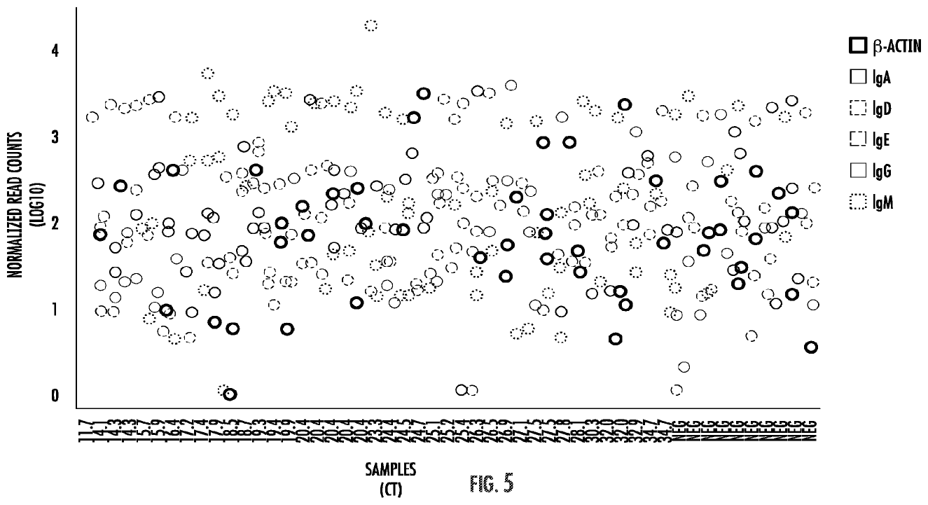

The present inventors first determined whether the standard RASL-seq assay, which does not involve RNA extraction, was compatible with analysis of COVID-19 patient mRNAs present in NP swab specimens, a matrix not previously analyzed using an oligonucleotide probe ligation technique. To this end, the present inventors utilized a large panel of RASL-seq probe sets designed to characterize human immune responses in a variety of settings (Supplemental Table 1 of Credle et al. (2020)). A pool of 1,736 probe sets targeting 240 genes, 154 of which were assessed by analysis of exon-exon junction usage, were included in a standard RASL-seq assay with oligo-dT coated magnetic beads for capture of polyadenylated mRNA transcripts. In this experiment, an average of 727 (±46) correctly paired probe sets, corresponding to 108 (±4) genes, were sequenced at least 10 times in a given sample. The present inventors observed very high reproducibility among technical replicates (average R2=0.95±0.03, FIG. 4). High levels of housekeeping genes were detected as expected, and patterns of immunoglobulin gene expression could be reliably measured and were consistent among patients even with very different SARS-COV-2 viral loads (determined by RT-qPCR, FIG. 5). These findings indicated that cRASL-seq analysis of non-polyadenylated, pathogen-associated RNA molecules might also be possible using unextracted NP swab specimens.

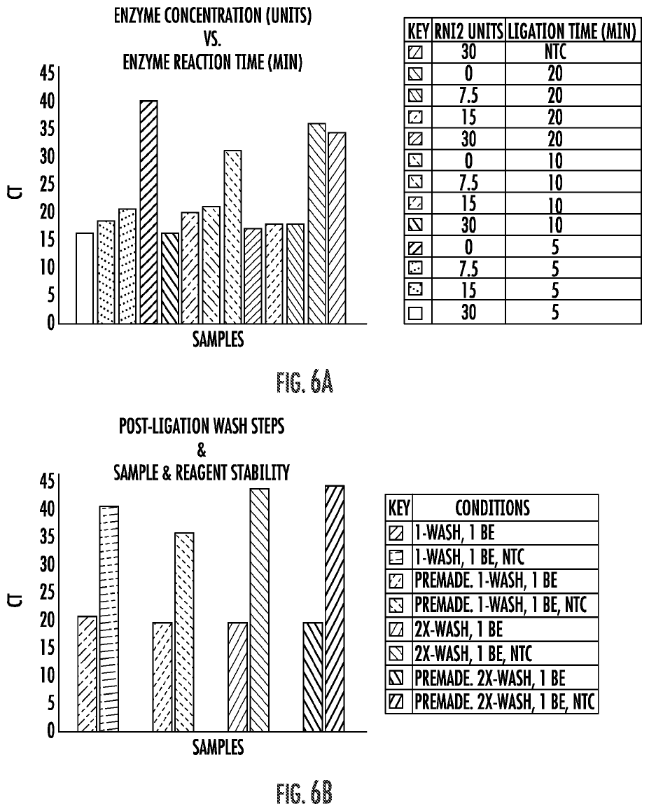

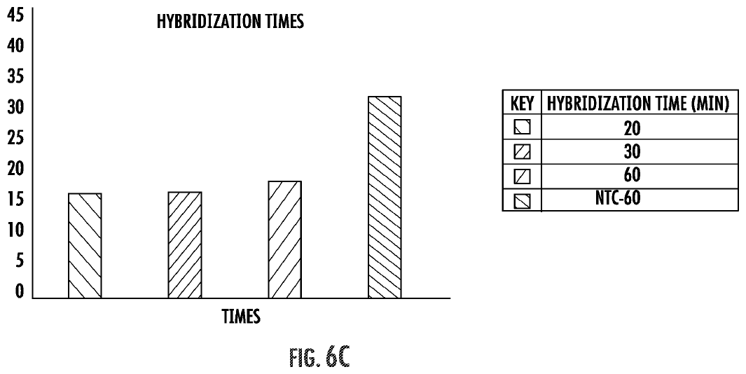

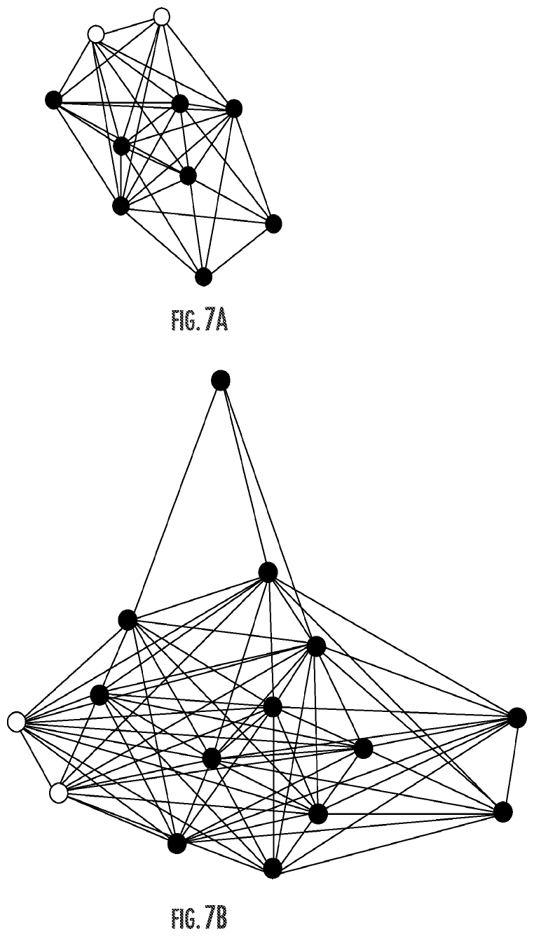

The present inventors determined the dynamic range of the cRASL-seq assay for detection of from 101 to 108 spiked-in target RNA molecules, observing exceptional linear performance over this range (FIG. 1D, R2=0.98). To make the cRASL-seq protocol as fast and inexpensive as possible, the present inventors performed extensive optimization to minimize the time and reagents required for each step (FIG. 1C, FIG. 6A-6C), without compromising assay sensitivity.

The present inventors next tested the performance of the cRASL-seq assay in detecting influenza A virus in blinded, previously characterized NP swabs obtained by the Johns Hopkins Center of Excellence for Influenza Research and Surveillance (JH-CEIRS). To detect influenza A, the present inventors designed a cRASL-seq probe set targeting a conserved sequence within the M-segment, according to the present inventors previously-established design principles.¹⁹ A PCR spike-in standard was added at a known concentration to enable precise calculation of the ligation product copy number. Upon specimen unblinding, the present inventors observed large numbers of reads mapping onto the correctly paired M-segment probe set in all samples that contained either H1N1 or H3N2 influenza virus (FIG. 1E). In contrast, either zero or a small number of reads mapped to the negative control samples, providing a large signal-to-noise ratio that ranged from 103 to 106.

The present inventors wondered to what extent the present inventors could multiplex target capture probes, since the present inventors expected magnetic capture bead capacity to limit the level of multiplexing achievable. In order to model increasing probe pool complexity, the present inventors serially diluted the influenza M-segment biotinylated capture probe (maintained at a standard 5 pM concentration) into a background of an irrelevant biotinylated capture probe at a concentration increasing up to the binding capacity of the streptavidin coated magnetic beads used in the assay. The present inventors compared the signal of the M-segment probe in the single-plex assay (no additional capture probe) to that observed in the model multiplexed assays. The present inventors observed >90% of the single-plex signal even in a background of 10,000-fold excess irrelevant capture probe (FIG. 1F). While the present inventors have not explicitly tested higher levels of ligation probe multiplexing in this study, previous RASL-seq studies have employed panels of >5,000 probe sets.²⁷ With appropriate design of non-interfering capture and ligation probe sets, the present inventors thus anticipate that the present inventors could achieve multiplexing of up to 10,000 probe sets, without technical artifacts.

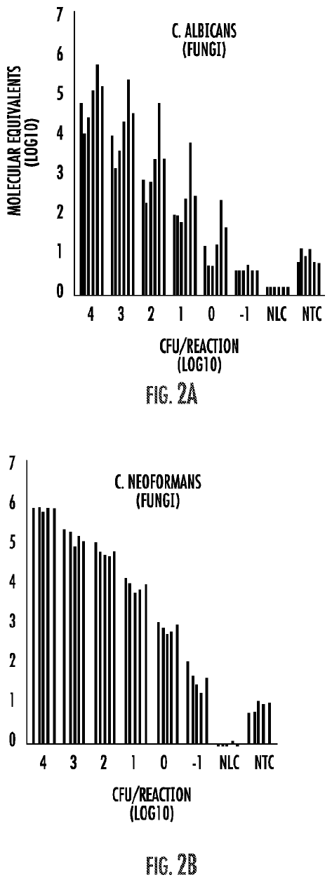

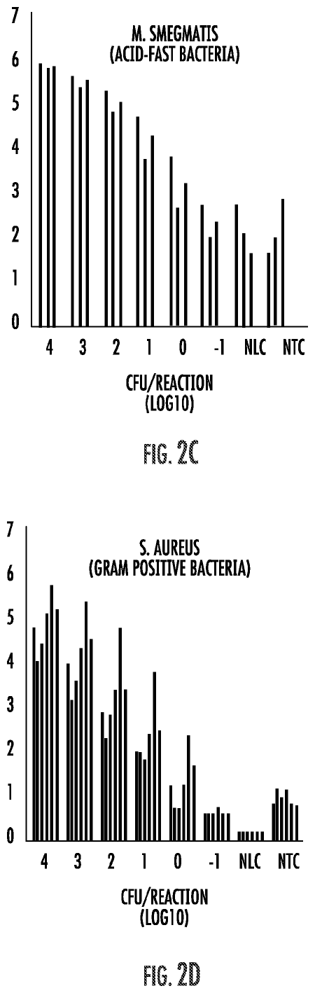

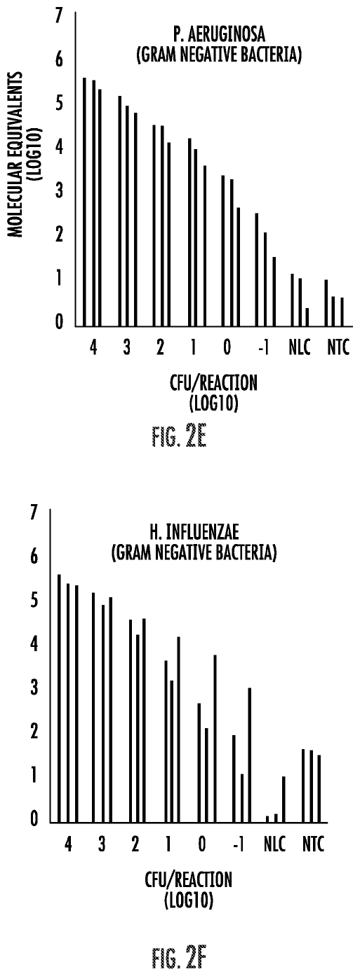

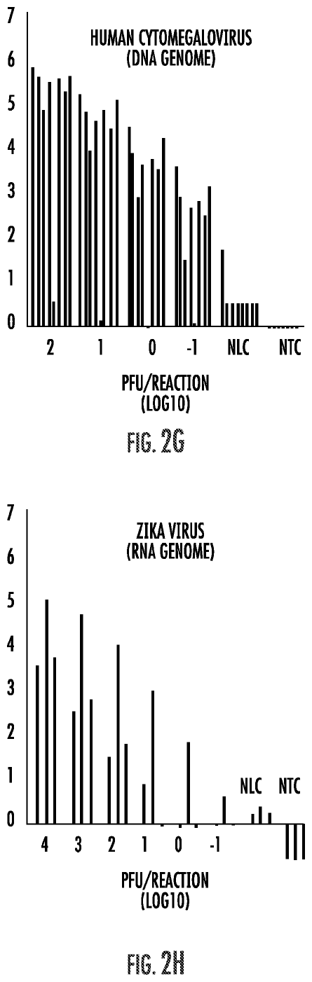

Having established that cRASL-seq could, in principle, be leveraged into a sophisticated infectious disease diagnostics platform, the present inventors next set out to determine whether a universal protocol could be employed for diverse classes of pathogens. Important human pathogens come from all kingdoms of life. The present inventors therefore tested the streamlined, extraction-free cRASL-seq protocol for detection of the following: fungal organisms (Candida albicans and Cryptococcus neoformans) using ITS and 26S/18S rRNA (FIG. 2A-B); acid fast bacteria (Mycobacterium smegmatis) (FIG. 2C), gram positive bacteria (Staphylococcus aureus) (FIG. 2D), and gram negative bacteria (Pseudomonas aeruginosa and Haemophilus influenzae) using 16S IRNA (FIG. 2E-F); DNA virus (Human cytomegalovirus) using pp65, US34, UL5, and UL22A mRNA (FIG. 2G); and RNA virus (Zika virus) using genomic RNA (FIG. 2H). Each organism was spiked into a separate reaction in serial dilution. The combined pool of 116 probes targeting all the RNAs were tested together in each reaction (Table 3). Negative control reactions included full reactions without any added organism (“no template control”, NTC), as well as reactions containing the organisms but lacking the Rnl2 enzyme during the ligation step (“no ligase control”, NLC). The present inventors considered an organism detected whenever the sum of the probes' normalized read counts was 10-fold higher than the corresponding normalized read counts from the NTC sample. In each case, the present inventors observed a strong linear correlation between normalized read counts and organism input amount across several logs of abundance, down to limits of detection that ranged from ˜1.5 to ˜150 colony or plaque forming units per milliliter (Table 2). cRASL-seq can therefore be used to detect a broad range of pathogens with clinically relevant sensitivity, using a universal, nucleic acid extraction free protocol.

TABLE 2

| | Limit of | |
|---|---|---|
| Organism | Detection | |
| C. albicans | 150 | cfu/mL |
| C. neoformans | 1.5 | cfu/mL |
| M. smegmatis | 150 | cfu/mL |
| S. aureus | 150 | cfu/mL |
| P. aeruginosa | 1.5 | cfu/mL |
| H. influenza | 1.5 | cfu/mL |
| Human | 1.5 | pfu/mL |
| cytomegalovirus | | |
| Zika virus | 15 | pfu/mL |

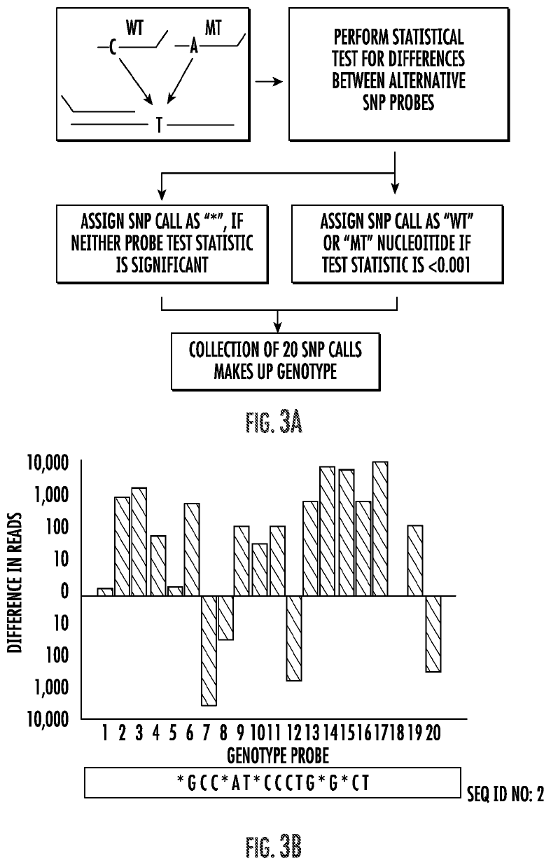

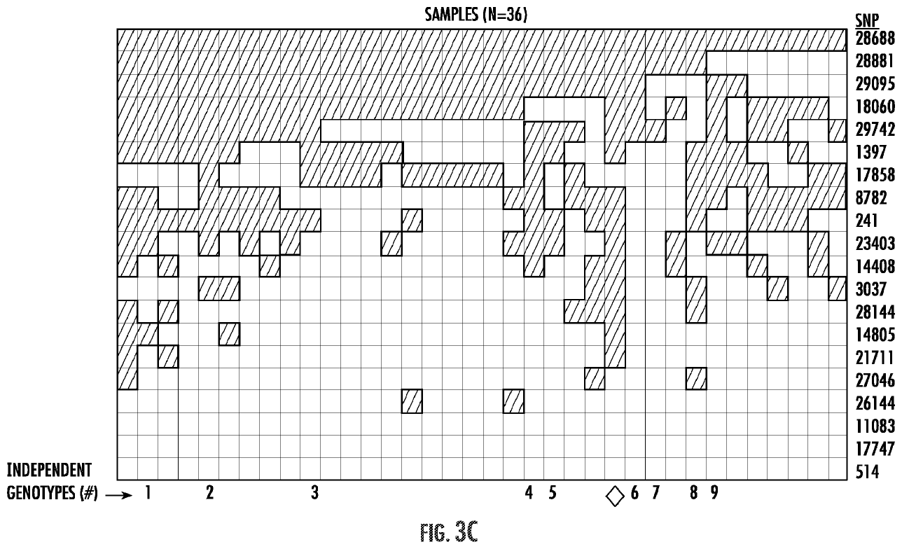

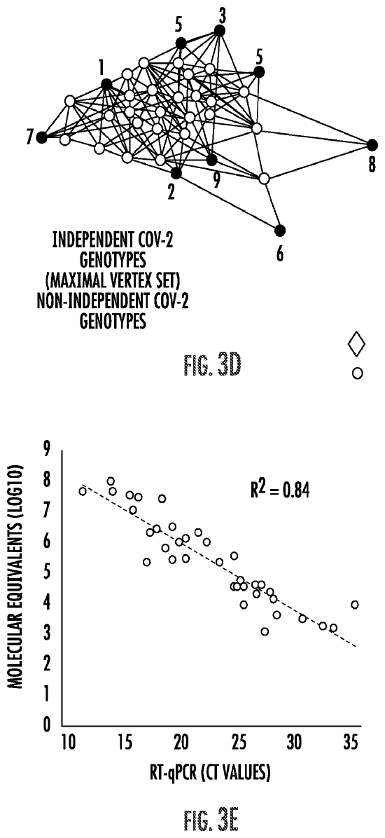

Analysis of pathogen single nucleotide polymorphisms (SNPs) has utility for distinguishing closely related organisms (e.g., human and zoonotic brucellosis), in tracing chains of viral transmission, and for detecting clade-specific differences in virulence. The present inventors therefore tested the ability of cRASL-seq probes to directly genotype SNPs from SARS-COV-2 RNA in COVID-19 patient NP swab specimens. Genotyping probe sets were designed to share a single 3′ acceptor probe, which could pair with two alternative 5′ phospho-donor probes corresponding to the alternative genotypes (FIG. 3A, Supplemental Table S3 of Credle et al. (2020) f). The present inventors placed the SNP recognition site in the center of the phosphodonor probe to maximally destabilize binding of the mismatched probe. Biotinylated capture probes were also designed to anneal within 200 nt of each genotyping probe set, to allow for some level of RNA degradation. In a proof-of-concept study, the present inventors designed genotyping probes for the 20 most entropic SARS-CoV-2 SNPs reported in the Nextstrain database²⁸ (queried on Mar. 16, 2020). These 20 SNPs span the majority of the SARS-COV-2 genome, ranging from genomic position 241 to 29,095.

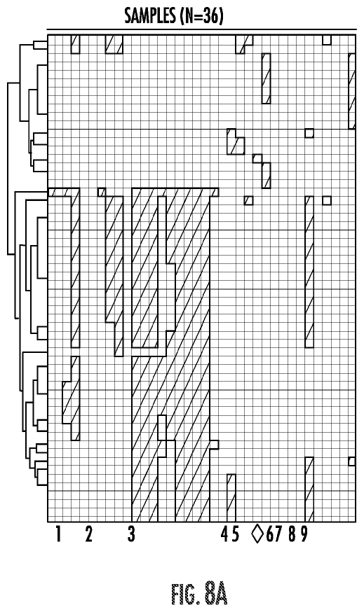

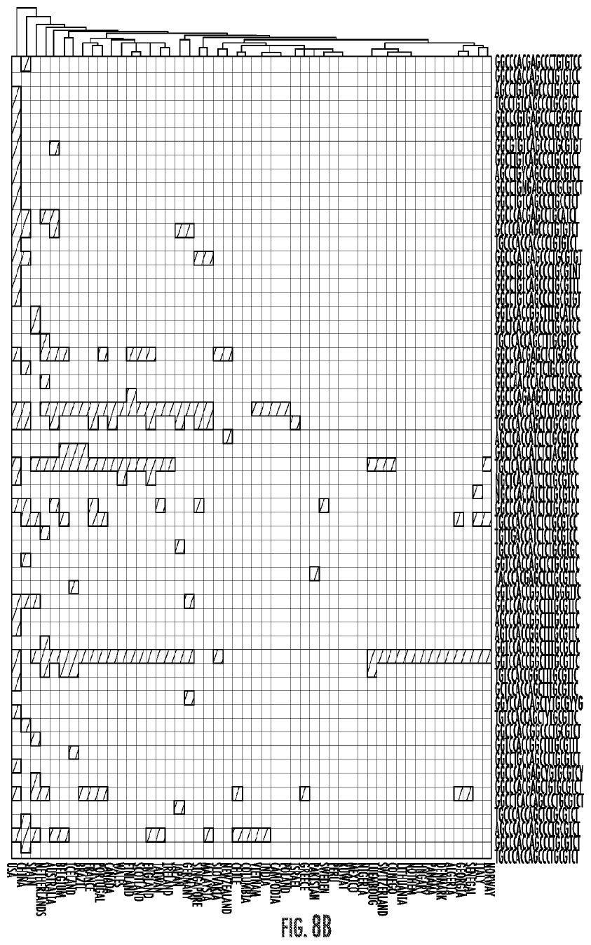

For each SNP, the number of reads from the reference (“wildtype”) probe sequence is compared against the number of reads obtained from the non-reference (“mutant”) probe sequence. If one of the probe sets is preferentially incorporated into the ligation product (fold-difference>1.5: p-value<0.001, binomial test), the base is called and assigned to the position. If the probes do not have sufficient reads or they are not significantly different, no base is called and a “wildcard” is assigned to the position. The string of assigned bases and wildcards are then compared to the strings of corresponding bases from each SARS-COV-2 genome deposited in the GISAID database (FIG. 3A, FIG. 8). The genotyping assay was first tested using purified reference SARS-COV-2 gRNA. At the highest input concentration, 2×10⁵ copies per reaction, 14 of the 20 bases were called in both technical replicates, and these genotypes matched perfectly to the sequence of the isolate from which it originated (hCoV19/USA/WA1/2020|EPI_ISL_404895|20200119, FIG. 3B). As the input gRNA amount decreased, significantly fewer bases were called with high confidence. The present inventors assessed the reproducibility of the assay by testing 8 NP swab specimens in duplicate and comparing the results (FIG. 7). All technical replicates agreed with each other at a cutoff of 5 SNPs called.

The present inventors used the 20 SNP SARS-COV-2 genotyping panel to analyze 40 NP swab specimens obtained from patients with RT-qPCR proven COVID-19. Of these, 5 or more SNPs could be called in 35 of the samples. These genotypes are displayed in FIG. 3C. To better understand the relationships among the observed genotypes, the present inventors used a network graph approach in which genotypes (nodes) are linked (share a connection) if they do not differ in any of the called SNPs (FIG. 3D). Using this network analysis, and by calculating the maximal independent vertex set, the present inventors were able to conclude that the infections among these 35 cases could be attributable to at least 9 distinct SARS-CoV-2 ancestral lineages, and thus at least that many distinct chains of local transmission. The reference Washington State isolate was unconnected to the present inventors' Baltimore network. The observed genotypes could additionally be associated with geographic locations, based on their matches to sequenced isolates in the GISAID database (FIG. 8).

Finally, the present inventors wondered whether viral load could be simultaneously estimated using data from genotyping probe sets alone. After background subtraction using no ligase controls and seasonal coronavirus samples, a probe set targeting a SNP in the nucleocapsid gene reported positive values in all 40 PCR+ samples (100% detection sensitivity). Furthermore, FIG. 3E illustrates how well correlated these values are with RT-qPCR Ct values (R2=0.84), indicating that cRASL-seq can be used to simultaneously determine viral load and viral genotype at high sensitivity, high throughput and very low cost.

### Discussion

The present inventors have developed a generalized version of the RASL-seq technology, called “capture RASL-seq” or “cRASLseq”, and demonstrated its utility for highly multiplexed molecular analyses of pathogens and host responses directly from NP swab specimens, using a universal and streamlined protocol that does not require up-front nucleic acid extraction or reverse transcription. The cRASL-seq protocol can easily be performed manually in a biosafety cabinet, does not require centrifugation or vortex steps that risk aerosolizing virus, does not rely on any specialized equipment, and incorporates easily scalable sample barcoding to dramatically reduce per-sample sequencing cost and increase throughput. Though not implemented here, the simple workflow can be easily automated using liquid handling instrumentation. For relatively low complexity probe panels, for example a simple SARS-COV-2 panel, tens of thousands of sequencing reads per sample may provide sufficient sensitivity. A high output Illumina NovaSeq 6000 instrument run, which can generate up to 1010 single end reads, would therefore provide sufficient depth to analyze >100,000 samples at once. Another advantage of the cRASL-seq methodology is the extremely low per-probe assay concentration of 5 pM. A typical 100 nmol oligonucleotide synthesis scale will therefore yield a sufficient quantity of probe for tens of millions of 100 μl cRASL-seq tests, at a per-test probe cost below one cent. The major cost-driving components of the reaction are the ligase, polymerase and magnetic beads. Enzyme production could be scaled up to reduce costs, while less expensive streptavidin capture matrices may be adapted to replace the magnetic beads. At the scale of millions of tests, it is therefore feasible to reduce the per-test reagent and sequencing costs to below one US dollar.

Obtaining SARS-COV-2 genotype information as part of a large-scale surveillance effort would have key benefits. Capturing viral genetics could potentially identify chains of transmission, enabling more effective contact tracing and policymaking, while also detecting and tracking emerging clades with enhanced or diminished virulence. There is also intense investigation into the role that host genetics plays in COVID-19 disease severity. As these genotypes are defined, RASL-seq probes that distinguish host alleles could be additionally incorporated into the assay. In this study, the present inventors separated the cRASL-seq analysis of SARS-COV-2 and the RASL-seq analysis of host immune responses into two separate reactions. However, by designing noninterfering probe panels and balancing the proportion of streptavidin coated magnetic beads, versus oligo-dT coated magnetic beads, it should be straightforward to perform the two assays simultaneously in a single reaction.

The cRASL-seq methodology is not without limitations. While the present inventors have demonstrated a sensitivity comparable to single-plex RT-qPCR, the limits of detection are governed by overall sequencing depth, which can be reduced by consumption of reads from highly abundant ligation products (due to a high load of a co-infecting virus for example). However, since the ligation products are all amplified with a high degree of uniformity, simple RNA spike-in or PCR spike-in sequences can be used to determine the lower limit of detection sensitivity for each reaction. Analysis of host transcripts can also be used to assess sample acquisition sufficiency, a known source of false negative test results.²⁹ Another important concern for COVID-19 molecular diagnostics is the turnaround time. When NGS is used to read out the cRASL-seq assay, the testing turnaround time is unlikely to be less than ˜24 hours with currently available instrumentation. Per-sample sequencing cost considerations will favor analysis of large sample batches, which could further increase turnaround times. For large-scale regional and national level surveillance purposes however, an occasional one to two days of self-quarantine while awaiting test results may be acceptable, given the costs and limitations associated with alternative methods. Faster, non-NGS-based readouts of cRASL probe ligation products may also be developed. For example, isothermal amplification, followed by array or test-strip hybridization may prove more applicable in the point-of-care setting. Regardless of the readout, a robust, rapidly reconfigurable, multiplexed, inexpensive and high sample throughput platform for molecular surveillance, such as the one described here, will facilitate curbing the COVID-19 pandemic and preventing future outbreaks from becoming pandemics.

### REFERENCES

1. Morens, D. M. & Fauci, A. S. Emerging Infectious Diseases: Threats to Human Health and Global Stability. PLOS Pathogens 9, e1003467 (2013).
2. Paules, C. I., Eisinger, R. W., Marston, H. D. & Fauci, A. S. What Recent History Has Taught Us About Responding to Emerging Infectious Disease Threats. Annals of Internal Medicine 167, 805-811 (2017).
3. Kandathil, A. J. et al. Presence of Human Hepegivirus-1 in a Cohort of People Who Inject Drugs. Ann Intern Med 167, 1-7 (2017).
4. Liu, J. et al. Development of a TaqMan Array Card for acute-febrile-illness outbreak investigation and surveillance of emerging pathogens, including Ebola virus. J Clin Microbiol 54, 49-58 (2016).
5. Hercik, C. et al. A Combined Syndromic Approach to Examine Viral, Bacterial, and Parasitic Agents among Febrile Patients: A Pilot Study in Kilombero, Tanzania. The American Journal of Tropical Medicine and Hygiene 98, 625-632 (2018).
6. Leber, A. L. et al. Multicenter evaluation of the BioFire FilmArray meningitis encephalitis panel for the detection of bacteria, ciruses and yeast in cerebrospinal fluid specimens. J Clin Microbiol (2016).
7. Buss, S. N. et al. Multicenter evaluation of the BioFire FilmArray gastrointestinal panel for etiologic diagnosis of infectious gastroenteritis. J Clin Microbiol 53, 915-925 (2015).
8. Srivatsan, S. et al. Preliminary support for a “dry swab, extraction free” protocol for SARS-COV-2 testing via RT-qPCR. bioRxiv (2020).
9. Schmid-Burgk, J. L. et al. LAMP-Seq: Population-Scale COVID-19 Diagnostics Using a Compressed Barcode Space. bioRxiv (2020).
10. National Institute of Allergy and Infectious Diseases, Vol. 2018 (2016).
11. Xu, M. et al. Comparative Diagnosis of Human Bocavirus 1 Respiratory Infection With Messenger RNA Reverse-Transcription Polymerase Chain Reaction (PCR), DNA Quantitative PCR, and Serology. J Infect Dis 215, 1551-1557 (2017).
12. Graf, E. H. et al. Unbiased detection of respiratory viruses by use of RNA sequencing-based metagenomics: a systematic comparison to a commercial PCR panel. J Clin Microbiol 54, 1000-1007 (2016).
13. Murphy, S. C. et al. Real-Time Quantitative Reverse Transcription PCR for Monitoring of Blood-Stage Plasmodium falciparum Infections in Malaria Human Challenge Trials. The American Journal of Tropical Medicine and Hygiene 86, 383-394 (2012).
14. Backstedt, B. T. et al. Efficient detection of pathogenic leptospires using 16S ribosomal RNA. PLOS One 10, e0128913 (2015).
15. Tsalik, E. L. et al. Host gene expression classifiers diagnose acute respiratory illness etiology. Sci Transl Med 8, 322ra311 (2016).
16. Woods, C. W. et al. A host transcriptional signature for presymptomatic detection of infection in humans exposed to influenza H1N1 or H3N2. PloS one 8, e52198(2013).
17. Ahn, S. H. et al. Gene expression-based classifiers identify Staphylococcus aureus infection in mice and humans. PloS one 8, e48979 (2013).
18. Esbin, M. N. et al. Overcoming the bottleneck to widespread testing: A rapid review of nucleic acid testing approaches for COVID-19 detection. RNA (2020).
19. Larman, H. B. et al. Sensitive, multiplex and direct quantification of RNA sequences using a modified RASL assay. Nucleic Acids Res 42, 9146-9157 (2014).
20. Joel J. Credle, C. Y. I., Tiezheng Yuan, Erick R. Scott, Rachael E. Workman, Yunfan Fan, Franck Housseau, Nicolas Llosa, W. Robert Bell, Heather Miller, Sean X. Zhang, Winston Timp & H. Benjamin Larman Multiplexed analysis of fixed tissue RNA using Ligation in situ Hybridization. Nucleic acids research gkx471. doi: 10.1093/nar/gkx471 (2017).
21. Zhang, P. et al. Multiplex ligation-dependent probe amplification (MLPA) for ultrasensitive multiplexed microRNA detection using ribonucleotide-modified DNA probes. Chem Commun (Camb) 49, 10013-10015 (2013).
22. Nandakumar, J. & Shuman, S. How an RNA ligase discriminates RNA versus DNA damage. Mol Cell 16, 211-221 (2004).
23. Nandakumar, J., Shuman, S. & Lima, C. D. RNA ligase structures reveal the basis for RNA specificity and conformational changes that drive ligation forward. Cell 127, 71-84 (2006).
24. Li, H., Qiu, J. & Fu, X. D. RASL-seq for massively parallel and quantitative analysis of gene expression. Curr Protoc Mol Biol Chapter 4, Unit 4 13 11-19 (2012).
25. Credle, J. J. et al. Multiplexed analysis of fixed tissue RNA using Ligation in situ Hybridization. Nucleic Acids Res 45, e128 (2017).
26. Yeakley, J. M. et al. A trichostatin A expression signature identified by TempO-Seq targeted whole transcriptome profiling. PLOS One 12, e0178302 (2017).
27. Yi, J. et al. JMJD6 and U2AF65 co-regulate alternative splicing in both JMJD6 enzymatic activity dependent and independent manner. Nucleic Acids Res 45, 3503-3518 (2017).
28. Hadfield, J. et al. Nextstrain: real-time tracking of pathogen evolution. Bioinformatics 34, 4121-4123 (2018).
29. Tang, Y. W., Schmitz, J. E., Persing, D. H. & Stratton, C. W. The Laboratory Diagnosis of COVID-19 Infection: Current Issues and Challenges. J Clin Microbiol (2020).
30. Metsky, H. C. et al. Capturing sequence diversity in metagenomes with comprehensive and scalable probe design. Nature Biotechnology 37, 160-168 (2019).
31. Steinegger, M. & Soding, J. MMseqs2 enables sensitive protein sequence searching for the analysis of massive data sets. Nat Biotechnol 35, 1026-1028 (2017).
32. Uhteg, K. et al. Comparing the analytical performance of three SARS-COV-2 molecular diagnostic assays. J Clin Virol 127, 104384 (2020).

1. A method for forming a target ribonucleic acid (RNA) proxy in a sample comprising the steps of:
(a) contacting a sample with one or more multi-partite probes that hybridize to a target RNA, wherein the one or more multi-partite probes comprise (i) a target capture probe, (ii) a 3′ acceptor probe and (iii) a 5′ phosphorylated donor probe;
(b) incubating the sample of step (a) under conditions that allow hybridization of the one or more multi-partite probes to target RNA present in the sample;
(c) immobilizing the target capture probes on a solid support;
(d) washing away unbound multi-partite probes; and
(e) ligating the acceptor probes and donor probes to form a target RNA proxy.

2. The method of claim 1, further comprising amplifying the target RNA proxy, wherein the 3′ acceptor probe and the 5′ phosphorylated donor probe comprise amplification primer binding sites.

3. The method of claim 1, further comprising detecting the target RNA by one or more of sequencing, quantitative PCR, microarray hybridization, toe-hold amplification, and loop-mediated isothermal amplification.

4. The method of claim 2, wherein the amount of target RNA is quantified.

5. The method of claim 1, wherein the 3′ acceptor probe comprises at least one 3′ terminal ribonucleotide.

6. The method of claim 5, wherein the 3′ acceptor probe comprises a 3′ terminal diribonucleotide.

7. The method of claim 1, wherein the target RNA is a pathogen-associated RNA.

8-14. (canceled)

15. A method for forming a target pathogen-associated RNA proxy in a sample obtained from a patient suspected of being infected with a pathogen comprising the steps of:
(a) contacting a sample obtained from the patient with one or more multi-partite probes that hybridize to a target pathogen-associated RNA, wherein the one or more multipartite probes comprise (i) a target capture probe, (ii) a 3′ acceptor probe and (iii) a 5. phosphorylated donor probe;
(b) incubating the sample of step (a) under conditions that allow hybridization of the one or more multi-partite probes to target pathogen-associated RNA present in the sample;
(c) immobilizing the target capture probes on a solid support;
(d) washing away unbound multi-partite probes; and
(e) ligating the acceptor probes and donor probes to form a target pathogen-associated RNA proxy.

16. The method of claim 15, further comprising amplifying the target pathogen-associated RNA proxy, wherein the 3′ acceptor probe and the 5′ phosphorylated donor probe comprise amplification primer binding sites.

17. The method of claim 15, further comprising detecting the target pathogen-associated RNA by one or more of sequencing, quantitative PCR, microarray hybridization, toe-hold amplification, and loop-mediated isothermal amplification.

18-21. (canceled)

22. A method for detecting pathogen contamination of food products or fomites comprising the steps of:
(a) contacting a sample obtained from a food product or fomite with one or more multi-partite probes that hybridize to a target pathogen-associated RNA, wherein the one or more multi-partite probes comprise (i) a target capture probe, (ii) a 3′ acceptor probe and (iii) a 5′ phosphorylated donor probe;
(b) incubating the sample of step (a) under conditions that allow hybridization of the one or more multi-partite probes to target pathogen-associated RNA present in the sample;
(c) immobilizing the target capture probes on a solid support;
(d) washing away unbound multi-partite probes;
(e) ligating the acceptor probes and donor probes to form a target pathogen-associated RNA proxy; and
(f) detecting the target pathogen-associated RNA proxy to identify the pathogen.

23. The method of claim 22, wherein detecting step (f) comprises sequencing, quantitative PCR, microarray hybridization, toe-hold amplification, and loop-mediated isothermal amplification.

24. The method of claim 22, wherein the amount of pathogen is quantified.

25. The method of claim 22, wherein the 3′ acceptor probe and/or the 5′ phosphorylated donor probe comprise alternative SNPs of the target pathogen-associated RNA to enable genotype identification.

26. (canceled)

27. A method for detecting a severe acute respiratory syndrome coronavirus 2 (SARS-COV-2) infection in a patient comprising the steps of:
(a) contacting a patient sample with one or more multi-partite probes that hybridize to a target SARS-COV-2-associated RNA, wherein the one or more multi-partite probes comprise (i) a target capture probe, (ii) a 3′ acceptor probe and (iii) a 5′ phosphorylated donor probe;
(b) incubating the sample of step (a) under conditions that allow hybridization of the one or more multi-partite probes to target SARS-COV-2-associated RNA present in the sample;
(c) immobilizing the target capture probes on a solid support;
(d) washing away unbound multi-partite probes;
(e) ligating the acceptor probes and donor probes to form a target SARS-CoV-2-associated RNA proxy; and
(f) detecting the target SARS-COV-2-associated RNA proxy, thereby detecting a SARS-COV-2 infection in the patient.

28. The method of claim 27, wherein detecting step (f) comprises sequencing, quantitative PCR, microarray hybridization, toe-hold amplification, and loop-mediated isothermal amplification.

29-32. (canceled)

33. The method of claim 27, wherein the one or more multi-partite probes comprise one or more of SEQ ID NOS:171-180.

34. The method of claim 30, wherein (i) the 3′ acceptor probe comprise one or more of SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, SEQ ID NO:159, SEQ ID NO:161, SEQ ID NO:163, SEQ ID NO:165, SEQ ID NO:167, and SEQ ID NO:169; (ii) the 5′ phosphorylated donor probe comprises one or more of SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, SEQ ID NO:160, SEQ ID NO:162, SEQ ID NO:164, SEQ ID NO:166, SEQ ID NO:168, SEQ ID NO:170.

35. The method of claim 27, wherein the target capture probe comprises one or more of SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, and SEQ ID NO:200.

36. The method of claim 1, wherein the labeled target capture probe comprises biotin, diogexin, acrydite, haloalkane, or click chemistry.

37-43. (canceled)
